(19) United States
(12) Patent Application Publication
(10) Pub. No.: US 2011/0129924 A1
(43) Pub. Date: Jun. 2, 2011

(54) Porous Polymeric Articles

(75) Inventors: Jackie Y. Ying, Singapore (SG); Edwin Pei Yong Chow, Singapore (SG); Jeremy Ming Hock Loh, Singapore (SG); Karl Schumacher, Singapore (SG)

(73) Assignee: Agency for Science, Technology and Research, Connexis (SG)

(21) Appl. No.: 12/308,960

(22) PCT Filed: Sep. 12, 2006
(86) PCT No.: PCT/US2006/035610

§ 371 Date: Jun. 24, 2009

Related U.S. Application Data
(60) Provisional application No. 60/818,336, filed on Jul. 5, 2006.

(51) Int. Cl. C12N 5/071

(52) U.S. Cl. 435/396

(57) ABSTRACT

Porous polymeric articles, and more specifically, porous polymeric articles for tissue engineering and organ replacement, are described. In some embodiments, methods described herein include use of a polymer-solvent system (e.g., phase inversion) to generate porosity in a structure. The process may include formation of a structure precursor material including a first crosslinkable component and a second component that can be precipitated in a precipitation medium. The structure precursor material may be shaped into a three-dimensional shape by a suitable technique such as three-dimensional printing. Upon shaping of the structure precursor material, at least a portion of the first component may be crosslinked. The structure may then be contacted with a precipitation medium to remove the precursor solvent from the structure, which can cause the second polymer component to precipitate and form a porous structure containing a network of uniform pores. In some embodiments, the porous structure is constructed and arranged for use as a template for ultrafiltration, cell growth, and/or for forming complex, biomimetic, porous biohybrid organs, where living cells can be immobilized and perform their normal physiological functions.

### FIELD OF INVENTION

The present invention relates generally to porous polymeric articles, and more specifically, to porous polymeric articles for tissue engineering and organ replacement.

### BACKGROUND

Tissue engineering and organ transplantation are principally concerned with the replacement of tissue and organs that have lost function due to injury or disease. In one approach toward this goal, organs are transplanted into a patient. However, the side effects of transplantation can be unpleasant, and can compromise the health of the organ recipient. In another approach, cells are cultured in vitro on biodegradable polymeric scaffolds to form tissues or neo organs that are then implanted into the body at the necessary anatomical site.

Several techniques have been proposed for forming scaffolds for tissue growth. For instance, U.S. Patent Publication No. 2002/0182241, entitled “Tissue Engineering of Three-Dimensional Vascularized Using Microfabricated Polymer Assembly Technology,” by Borenstein et al., describes two-dimensional templates that are fabricated using high-resolution molding processes. These templates are then bonded to form three-dimensional scaffold structures with closed lumens. U.S. Pat. No. 6,176,874, entitled “Vascularized Tissue Regeneration Matrices Formed by Solid Free Form Fabrication Techniques,” by Vacanti et al., describes solid free-form fabrication methods used to manufacture devices for allowing tissue regeneration and for seeding and implanting cells to form organ and structural components. U.S. Patent Publication No. 2003/0069718, entitled “Design Methodology for Tissue Engineering Scaffolds and Biomaterial Implants,” by Hollister et al., describes anatomically shaped scaffold architectures with heterogeneous material properties, including interconnecting pores.

Despite the above efforts, significant developments in connection with many internal, physical structures, especially those of hollow and epithelial organs, has been limited, and improvements are needed. Particularly, new methods for fabricating articles having small and uniform pore sizes for tissue engineering and organ replacement would be beneficial.

### SUMMARY OF THE INVENTION

Porous polymeric articles, and more specifically, porous polymeric articles for tissue engineering and organ replacement are provided. In one aspect, a series of methods of fabricating a structure for use as a template for cell growth are provided. In one embodiment, the method comprises dissolving at least first and second polymer components in a precursor solvent to form a structure precursor material, shaping the structure precursor material into a structure suitable for use as a template for cell growth, crosslinking the first polymer component, and removing at least a portion of the precursor solvent from the structure, thereby forming a plurality of pores in the structure.

In another embodiment, a method of fabricating a structure for use as a template for cell growth is provided. The method comprises providing a structure precursor material comprising at least first, second, and third components, shaping the structure precursor material into a structure suitable for use as a template for cell growth, crosslinking the first component, precipitating the second component in a precipitation medium, and removing the third component from the structure in the precipitation medium, thereby forming a plurality of pores in the structure.

In another embodiment, a method of fabricating a structure for use as a template for cell growth is provided. The method comprises mixing at least first and second polymer components in a precursor solvent to form a homogeneous structure precursor material, wherein the first and second polymer components and the precursor solvent are miscible at 25 degrees Celsius and 1 atm, printing the structure precursor material to form a three-dimensional structure suitable for use as a template for cell growth, and removing the precursor solvent from the structure, thereby forming a plurality of pores in the structure.

In another embodiment, a method of fabricating a structure for use as a template for cell growth is provided. The method comprises forming a cell growth template precursor structure comprising at least first and second polymer components and a fluid carrier, crosslinking the first polymer component thereby forming a self-supporting structure, and removing at least a portion of the fluid carrier from the self-supporting structure, thereby forming a plurality of pores in the structure suitable for templated cell growth, wherein the porous structure is formed in a shape suitable for templated cell growth.

In another embodiment, a method of fabricating a structure for use as a template for cell growth is provided. The method comprises dissolving at least first and second polymer components in a precursor solvent to form a structure precursor material, shaping the structure precursor material into a structure suitable for use as a template for cell growth, exposing the structure precursor material to UV radiation, and removing at least a portion of the precursor solvent from the structure, thereby forming a plurality of pores in the structure.

In another aspect, an article for use as a template for cell growth is provided. The article comprises a structure comprising at least one wall defining a cavity, and a plurality of pores having an average pore size of less than or equal to 20 microns formed in at least a portion of the wall, wherein no more than about 5% of all pores deviate in size from the average pore size of the plurality of pores by more than about 20%, wherein the structure is constructed and arranged for use as a template for cell growth.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows a process for forming a three-dimensional structure according to one embodiment of the invention;

FIGS. 2A and 2B show schematic diagrams of a three-dimensional printing process for forming three-dimensional structures, according to one embodiment of the invention;

FIG. 3 shows a schematic diagram of a phase inversion process for forming a porous structure, according to one embodiment of the invention;

FIG. 4 shows a schematic diagram of a filtration process for characterizing pore size and molecular weight cutoff of a porous structure, according to one embodiment of the invention;

FIG. 5 shows a plot of water flux as a function of pressure for various porous membranes, according to one embodiment of the invention;

FIG. 6A-6F shows SEM micrographs of porous PS-FC membranes, according to one embodiment of the invention;

FIG. 7 shows PEG rejection curves for the porous membranes of FIGS. 6A-6F as a function of PEG molecular weight, according to one embodiment of the invention;

FIG. 8A shows MDCK cells adhered to a commercial membrane without the use of adhesion proteins, according to one embodiment of the invention; and

FIGS. 8B-8C show MDCK cells adhered to a PS-FC membrane without the use of adhesion proteins, according to one embodiment of the invention.

### DETAILED DESCRIPTION

Porous polymeric articles, and more specifically, porous polymeric articles for tissue engineering and organ replacement, are described. In some embodiments, methods described herein include use of a polymer-solvent system (e.g., phase inversion) to generate porosity in a structure. The process may include formation of a structure precursor material including a first crosslinkable component and a second component that can be precipitated in a precipitation medium. The structure precursor material may be shaped into a three-dimensional shape by a suitable technique such as three-dimensional printing. Upon shaping of the structure precursor material, at least a portion of the first component may be crosslinked. The structure may then be contacted with a precipitation medium to remove the precursor solvent from the structure, which can cause the second polymer component to precipitate and form a porous structure containing a network of uniform pores. In some embodiments, the porous structure is constructed and arranged for use as a template for ultrafiltration, cell growth, and/or for forming complex, biomimetic, porous biohybrid organs, where living cells can be immobilized and perform their normal physiological functions.

Advantageously, structures described herein may have attractive biofunctional characteristics such as uniform pores having a sharp molecular weight cut-off (MWCO), high filtration and diffusion fluxes, and good mechanical strength, biocompatibility and formability.

Although much of the description herein involves an exemplary application of the present invention related to using porous polymeric articles as scaffolds for tissue engineering and/or organ replacement, the invention and its uses are not so limited, and it should be understood that the invention can also be used in other settings such as for filtration, purification, and separation processes.

In some embodiments, structures described herein can be drawn, imaged, and/or scanned using a variety of tools, including computer-aided design (CAD) tools, high-resolution multi-section computed tomography (CT) scans, and/or three-dimensional scanners. For instance, for structures to be used for tissue engineering and/or organ replacement, a CT scan of a tissue and/or organ of a patient can be converted into a proper file format, and fed into a system that can produce the structures. A variety of techniques can be used to form the structures, as described in more detail below. These methods can, in some cases, control compositions and micro-architectures of the structures. Appropriate systems and techniques for fabricating structures for tissue engineering and/or organ replacement include, but are not limited to, three-dimensional printing (e.g., three-dimensional layering), multi-photon lithography, stereolithography (SLA), selective laser sintering (SLS) or laser ablation, ballistic particle manufacturing (BPM), laminated object manufacturing, and fusion deposition modeling (FDM). In certain preferred embodiments, structures are formed by three-dimensional printing. Other techniques for fabricating structures for tissue engineering and/or organ replacement can also be used. Such techniques can be combined with appropriate materials and/or steps to fabricate porous articles described herein.

In one embodiment, a three-dimensional printing technique is used to fabricate a porous article. The three-dimensional printing technique may include the use of a tool such as the Eden 260 Rapid Prototyping Tool (RPT). The Eden 260 RPT is a polymer dispensing system that can print droplets of a polymer precursor material and, if desired, a sacrificial material, using a piezoelectric-actuated nozzle. Using such tools, a three-dimensional image file can be processed and the image may be sliced into many layers. Each layer can then printed on top of each other, and at least a portion of the polymer precursor material can be polymerized and/or crosslinked.

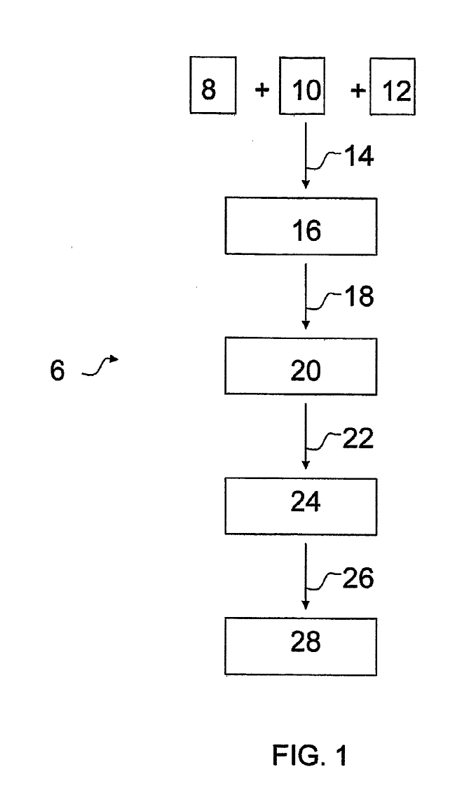

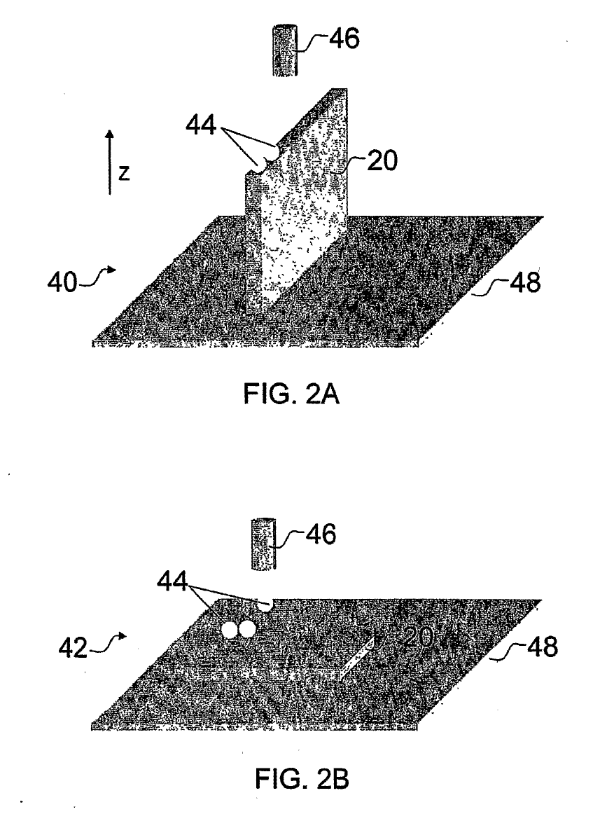

An example of a process for forming a three-dimensional structure is shown in FIG. 1. As shown in process 6, structure precursor material 16 to be shaped (e.g., into a template for cell growth) may include first component 8 comprising a monomer (e.g., a UV crosslinkable monomer) and second component 10 comprising a monomer/polymer that can be precipitated. The first and/or second components may be dissolved in solvent 12 (e.g., a third component or a fluid carrier). In some embodiments, step 14 of combining (e.g., mixing) the first and second components with the solvent may form a homogeneous solution. The structure precursor material can then be shaped in step 18 into a first precursor structure 20, which may be a cell growth template precursor structure. The shaping of the precursor structure material may take place by three-dimensional printing, as illustrated in FIGS. 2A and 2B, or by another suitable technique. For example, in the embodiment illustrated in FIGS. 2A and 2B, the structure precursor material (and a sacrificial material, if desired) can be dispensed droplet by droplet and layer by layer. Tools 40 and 42 can dispense droplets 44 of the same or different materials using one or more nozzles 46. Droplets 44 may be printed to form first precursor structure 20, supported by substrate holder 48. As shown in FIG. 2A, first precursor structure 20 can be formed by a vertical printing process; FIG. 2B shows horizontal printing of first precursor structure 20. Optionally, after each layer of material has been dispensed, a roller may be used to smooth out the surface. The first component of the structure precursor material (e.g., the crosslinkable monomer) may be polymerized (and/or crosslinked) in step 22 of FIG. 1, which may include, for example, exposure of the structure precursor material to UV radiation or any suitable source that can cause polymerization and/or crosslinking of at least a portion of the material. This process may be repeated until the formation of second precursor structure 24, which may be in the form of a solid or semi-solid structure (e.g., a self-supporting structure). In other embodiments, several or all layers of the structure precursor material may be dispensed before polymerization and/or crosslinking of at least one component of the structure precursor material. In some cases, the second component does not substantially polymerize and/or crosslink upon exposure to UV radiation. The structure can then be contacted with a precipitation medium in step 26 and at least a portion of the precursor solvent may be removed in the medium. This process can cause the second component to precipitate and form porous structure 28 containing a network of uniform pores. Alternatively, in some embodiments, first precursor structure 20 may be contacted with a precipitation medium in step 26, followed by polymerization and/or crosslinking of a component of the precursor structure to form porous structure 28. The porous structure may be formed in a shape suitable for templated cell growth.

In some cases, porous structure 28 may be designed to include open areas (e.g., pores and/or cavities). During fabrication, the open areas may be filled with a sacrificial material. The sacrificial material and the precursor material may be dispensed by separate nozzles of a three-dimensional printer. After printing, the sacrificial material may be removed, for example, by dissolving the material in a solvent. Typically, a suitable sacrificial material includes one that is soluble in a solution that does not dissolve the structure precursor material. In some cases, the sacrificial material is not polymerizable and/or crosslinkable; however, in other cases, the sacrificial material is polymerizable and/or crosslinkable.

A further description of the phase inversion process is now described. As described herein, in some embodiments, fabrication of a structure can involve dissolving one or more components (e.g., a first and/or a second polymer component, which may include monomers and/or polymers) in an appropriate precursor solvent to form a structure precursor solution. In one particular embodiment, the first and second components are miscible at 25 degrees Celsius and 1 atm. The structure precursor solution can be shaped into the desired structure. For example, in one embodiment, the structure precursor solution is cast as a membrane or hollow fiber. In another embodiment, the structure precursor solution is shaped into a three-dimensional structure by a suitable technique, such as three-dimensional printing. Optionally, at least one component (e.g., a first polymer component) of the structure can be polymerized and/or crosslinked, e.g., by exposing the component to ultraviolet (UV) radiation. Polymerization and/or crosslinking may take place after all, or portions, of the structure has been shaped. In some cases, polymerization and/or crosslinking can cause solidification of portions of the structure. The structure can then be immersed in a precipitation medium (e.g., a solvent, also called a “non-solvent”, in the form of a liquid or a gas) that can precipitate at least one component (e.g., a second polymer component) of the structure. This process can cause separation of the precursor structure into a solid polymer and a liquid solvent phase including the precursor solvent. At least a portion of the precursor solvent may be removed in the precipitation medium, which can cause the second polymer component to precipitate and form a porous structure containing a network of uniform pores.

Parameters that affect the structure and properties of the desired structure may include the composition of the precipitation media, the component concentrations, the viscosity of the precursor material (which, in turn, may depend on the method used to shape/form the structure), the relative glass transition temperature and the viscosity ratio (or molecular weight ratio) of the components (e.g., if the precursor material includes more than one components), the temperature of the structure precursor material and/or precipitation medium, component molecular weight and solubility parameters (of the component(s), solvent, and precipitation medium), and the amount and type of precursor solvent. These factors can be varied to produce structures with, for example, a large range of pore sizes (e.g., from 0.01 to 20 microns), and, in some embodiments, with no more than about 5% of all pores deviating in size from the average pore size by more than about 20%, as described in more detail below.

Different methods of precipitation can be used to induce precipitation of a component of a structure precursor material. In some instances, precipitation of a component from a precursor structure can be caused by changing the concentration of the component in the precursor structure. For instance, in one embodiment, a precursor structure or precursor solution comprising a polymer component and a precursor solvent can be brought into contact with a precipitation medium. At least a portion of the precursor solvent can diffuse outwards into the precipitation medium and at least a portion of the precipitation medium can diffuse into the structure or precursor solution. After a given period of time, the exchange of the precursor solvent and precipitation medium can cause the precursor structure/solution to become thermodynamically unstable. As a result, demixing can occur and the polymer component can precipitate to form a solid network. Alternatively, in some cases the precipitation medium may be a gas (e.g., air, nitrogen, oxygen, and carbon dioxide) and evaporation of the precursor solvent can cause precipitation of a polymer component. In another embodiment, a polymer component dissolved in a solvent can solidify by a temperature change (e.g., upon cooling). This may be performed, for example, by reducing the temperature of the precursor structure/solution to below the glass transition temperature or the melting point of the polymer component to be precipitated.

The rate of precipitation of one component of the structure precursor material may be controlled by choosing appropriate compositions and/or conditions of the precipitation medium. For instance, it is known that the quicker a component is caused to precipitate, the finer is the dispersion of the precipitating phase. High rates of precipitation may occur by exposing the precipitating component to a precipitation medium having a very different solubility parameter than that of the component. The length of exposure of the component to the precipitation medium and the temperature difference between the two may also change the rate of precipitation. Accordingly, structural integrity and morphological properties of the final porous structure can be varied by controlling such parameters.

Materials suitable for use as a precipitation medium include, for example, liquids or gases that can cause at least one component of the structure precursor material to precipitate upon exposure to the medium. For example, a structure precursor material comprising polysulfone can be precipitated by exposure of the material to water, which acts as a suitable precipitation medium. A suitable precipitation medium for a structure precursor material may be chosen based on the solubility of the component to be precipitated in the precipitation medium, e.g., using known solubility properties of the materials or by simple experimentation. For instance, solubility parameters (e.g., Hildebrand parameters), as described in Barton, Handbook of Solubility Parameters, CRC Press, 1983, may be used to determine the likelihood of solubility of one component in another. Typically, chemical components having different values of solubility parameter are not soluble in one another. In certain embodiments, a structure precursor material component that is non reactive with, and precipitates upon exposure to, a precipitation medium is preferred. Accordingly, a structure precursor material component and a precipitation medium having different values of solubility parameter may be chosen. Those of ordinary skill in the art can also choose an appropriate structure precursor material component and/or precipitation medium by a simple screening test. One simple screening test may include mixing the structure precursor material component with the precipitation medium and determining whether the components react with and/or causes the precursor material component to precipitate. Varying conditions such as temperatures and concentrations of materials may be used in such an experimentation. Other simple tests can be conducted by those of ordinary skill in the art.

A variety of materials can be used to fabricate structures of the present invention. Materials used to form structures for tissue engineering and/or organ replacement may be biocompatible, and can include, for example, synthetic or natural polymers, inorganic materials, or composites of inorganic materials with polymers.

As described above, in some embodiments, structures described herein are formed of a structure precursor material that includes at least first and second polymer components. The first and second polymer components may be, for example, monomers that can be or a polymerized and/or crosslinked, or polymers that can be further polymerized and/or crosslinked by any suitable means. Sometimes, the first polymer component can be dissolved in the second polymer component (or in a solvent compatible with both polymer components) such that the component molecules interpenetrate one another. The structure precursor material may not stable thermodynamically, meaning that a demixing process may occur in the material. To increase the stability, it is often necessary to polymerize, crosslink or precipitate one or both polymer components. Accordingly, in one embodiment, a polymer component of a structure precursor material is substantially soluble in a precursor solvent but substantially insoluble in a precipitation medium, such that at least a portion of the component precipitates upon contact with the precipitation medium. In another embodiment, a polymer component of a structure precursor can be polymerized and/or crosslinked by a suitable technique, such as exposure to UV radiation, heat, and or a crosslinking agent. In yet another embodiment, polymer components (and/or a precursor solvent) are chosen at least partially based on their solubility in one another, e.g., using known solubility properties of the components or by simple experimentation. For instance, solubility parameters (e.g., Hildebrand parameters), as described in Barton, Handbook of Solubility Parameters, CRC Press, 1983, may be used to determine the likelihood of solubility of one component in another. Typically, chemical components having similar values of solubility parameter are soluble in one another. Those of ordinary skill in the art can also choose an appropriate polymer and/or solvent by, e.g., the likelihood of reactivity between the components and the solvent, and/or by a simple screening test. One simple screening test may include mixing the polymer components together, optionally with a precursor solvent, and determining whether the components react with one another and/or form a homogeneous solution. In certain embodiments, non-reactive components that form a miscible (e.g., homogeneous) solution at 25 degrees Celsius and 1 atm are preferred. Other simple tests can be conducted by those of ordinary skill in the art.

In some embodiments, a polymer component includes one or more photocurable (e.g., crosslinkable) polymers (or monomers). For instance, photocurable polymers may include ultra-violet or visible-light curable polymers. Particular materials include photocurable acrylic monomers, acrylic polymers, UV curable monomers, thermal curable monomers, polymer solutions such as melted polymers and/or oligomer solutions, poly methyl methacrylate, poly vinylphenol, benzocyclobutene, polyethylene oxide precursors terminated with photo-crosslinking end groups, one or more polyimides, and monomers of such polymers. In some cases, acrylate-based photo-polymers can include one or more components such as a sensitizer dye, an amine photo-initiator, and a multifunctional acrylate monomer. For example, pentaerythritol triacrylate (PETIA,) can form the backbone of the polymer network, N-methyldiethanolamine (MDEA) can be used as a photo-initiator, and Eosin Y (2-, 4-, 5-, 7-tetrabromofluorescein disodium salt) can be used as a sensitizer dye. This system is particularly sensitive in the spectral region from 450 to 550 nm, and can be used, for instance, in two-photon lithography involving a 1028 nm laser. In another example, an organic-inorganic hybrid such as ORMOCER® (Micro Resist Technology) can be used to fabricate structures described herein. This material can show high transparency in the visible and near infrared ranges, can contain a highly crosslinkable organic network, can incorporate inorganic components that may lead to high optical quality and high mechanical and thermal stability, and can be biocompatible for certain types of cells and/or cellular components. In yet another example, acrylate and epoxy polymers such as ethoxylated trimethylolpropane triacrylate ester and alkoxylated trifunctional acrylate ester can be used to form structures.

Structure precursor materials may additionally include one or more photoinitiators and/or crosslinkers for polymerization and/or crosslinking. Additionally, the structure precursor material may optionally be diluted in one or more solvents in order to decrease the viscosity of the material and to make it suitable for application, for example, in an ejection mechanism such as a three-dimensional printer.

In certain embodiments, photopolymerizable materials that are also biocompatible and water-soluble can be used to form structures for tissue engineering and/or organ replacement. A non-limiting example includes polyethylene glycol tetraacrylate, which can be photopolymerized with an argon laser under biologically compatible conditions, i.e., using an initiator such as triethanolamine, N-vinylpyrrolidone, and eosin Y. Similar photopolymerizable units having a poly(ethylene glycol) central block, extended with hydrolyzable oligomers such as oligo(d,l-lactic acid) or oligo(glycolic acid), and terminated with acrylate groups, may be used. Other polymerizable and/or crosslinkable polymers that polymerize or crosslink, for example, upon exposure to heat and/or chemical crosslinking agents may also be used.

Additional examples of polymer components that can be used to form structures described herein include but are not limited to: polyvinyl alcohol, polyvinylbutryl, polyvinylpyridyl, polyvinyl pyrrolidone, polyvinyl acetate, acrylonitrile butadiene styrene (ABS), ethylene-propylene rubbers (EPDM), EPR, chlorinated polyethylene (CPE), ethelynebisacrylamide (EBA), acrylates (e.g., alkyl acrylates, glycol acrylates, polyglycol acrylates, ethylene ethyl acrylate (EEA)), hydrogenated nitrile butadiene rubber (HNBR), natural rubber, nitrile butadiene rubber (NBR), certain fluoropolymers, silicone rubber, polyisoprene, ethylene vinyl acetate (EVA), chlorosulfonyl rubber, flourinated poly(arylene ether) (FPAE), polyether ketones, polysulfones, polyether imides, diepoxides, diisocyanates, diisothiocyanates, formaldehyde resins, amino resins, plyurethanes, unsaturated polyethers, polyglycol vinyl ethers, polyglycol divinyl ethers, poly(anhydrides), polyorthoesters, polyphosphazenes, polybutylenes, polycapralactones, polycarbonates, and protein polymers such as albumin, collagen, and polysaccharides, copolymers thereof, and monomers of such polymers. In certain embodiments, a polymer component is chosen based on its compatibility with a three-dimensional printing technique.

In one particular embodiment, a structure precursor material comprises the UV curable acrylic monomer comprising Objet FullCure™ 3D printing build material, which is available from Objet Geometries Inc. Upon exposure of a structure precursor material comprising the build material to UV radiation, at least a portion of the acrylic monomers may polymerize and/or crosslink to form a solid or semi-solid precursor structure. In this embodiment, the structure precursor material may additionally comprise a photoinitiator for polymerization, and may be diluted in one or more solvents, such as an alcohol, e.g., isopropropyl alcohol, ethanol, and/or methanol, or any other suitable solvent, in order to decrease the UV curable monomer viscosity and to make it suitable for application, for example, in an ejection mechanism such as a three-dimensional printer.

In some cases, a polymer precursor material includes a polymer that can precipitate upon exposure to a precipitation medium. In one particular embodiment, the polymer component is a polysulfone. Polysulfones include, for example, polyether sulfones, polyaryl sulfones (e.g., polyphenyl sulfone), polyalkyl sulfones, polyaralkyl sulfones, and the like.

Other polymers that may precipitate upon exposure to a precipitation medium that may be used as a structure precursor component include, but are not limited to, polyamines (e.g., poly(ethylene imine) and polypropylene imine (PPI)); polyamides (e.g., polyamide (Nylon), poly(ε-caprolactam) (Nylon 6), poly(hexamethylene adipamide) (Nylon 66)), polyimides (e.g., polyimide, polynitrile, and poly(pyromellitimide-1,4-diphenyl ether) (Kapton)); vinyl polymers (e.g., polyacrylamide, poly(2-vinyl pyridine), poly(N-vinylpyrrolidone), poly(methylcyanoacrylate), poly(ethylcyanoacrylate), poly(butylcyanoacrylate), poly(isobutylcyanoacrylate), poly(vinyl acetate), poly (vinyl alcohol), poly(vinyl chloride), poly(vinyl fluoride), poly(2-vinyl pyridine), vinyl polymer, polychlorotrifluoro ethylene, and poly(isohexylcynaoacrylate)); polyacetals; polyolefins (e.g., poly(butene-1), poly(n-pentene-2), polypropylene, polytetrafluoroethylene); polyesters (e.g., polycarbonate, polybutylene terephthalate, polyhydroxybutyrate); polyethers (poly(ethylene oxide) (PEO), poly(propylene oxide) (PPO), poly(tetramethylene oxide) (PTMO)); vinylidene polymers (e.g., polyisobutylene, poly(methyl styrene), poly(methylmethacrylate) (PMMA), poly(vinylidene chloride), and poly(vinylidene fluoride)); polyaramides (e.g., poly(imino-1,3-phenylene iminoisophthaloyl) and poly(imino-1,4-phenylene iminoterephthaloyl)); polyheteroaromatic compounds (e.g., polybenzimidazole (PBI), polybenzobisoxazole (PBO) and polybenzobisthiazole (PBT)); polyheterocyclic compounds (e.g., polypyrrole); polyurethanes; phenolic polymers (e.g., phenol-formaldehyde); polyalkynes (e.g., polyacetylene); polydienes (e.g., 1,2-polybutadiene, cis or trans-1,4-polybutadiene); polysiloxanes (e.g., poly(dimethylsiloxane) (PDMS), poly(diethylsiloxane) (PDES), polydiphenylsiloxane (PDPS), and polymethylphenylsiloxane (PMPS)); and inorganic polymers (e.g., polyphosphazene, polyphosphonate, polysilanes, polysilazanes); monomers of such polymers, as well as other polymers and monomers described herein.

Structures described herein may be hydrophobic or hydrophilic. Hydrophobic structures can be formed of hydrophobic polymers including, but not limited to, polypropylene, polyvinylidene fluoride, polyethylene, polyvinylidene fluoride, poly(tetrafluoroethylene). In some cases, at least a portion of a hydrophobic can be made hydrophilic, e.g., by surface modification. Hydrophilic polymers may also be used, as described herein.

A polymer component may be non-biodegradable or biodegradable (e.g., via hydrolysis or enzymatic cleavage). In some embodiments, biodegradable polyesters such as polylactide, polyglycolide, and other alpha-hydroxy acids can be used to form structures. By varying the monomer ratios, for example, in lactide/glycolide copolymers, physical properties and degradation times of the polymer can be varied. For instance, poly-L-lactic acid (PLLA) and poly-glycolic acid (PGA) exhibit a high degree of crystallinity and degrade relatively slowly, while copolymers of PLLA and PGA, PLGAs, are amorphous and rapidly degraded.

In some cases, biocompatible polymers having low melting temperatures are desired. Non-limiting examples include polyethylene glycol (PEG) 400 (melting temperature=4-80° C.), PEG 600 (melting temperature=20-25° C.), PEG 1500 (melting temperature=44-480° C.), and stearic acid (melting temperature=70° C.).

In some embodiments, a polymer precursor material can include a non-polymeric material. Non-limiting examples of such materials include organic and inorganic materials such as ceramics, glass, hydroxyapatite, calcium carbonate, buffering agents, as well as drug delivery carriers (e.g., gels), which can be solidified by application of an adhesive or binder.

In certain embodiments, additives can be added to a structure precursor material. Additives may, for instance, increase a physical (e.g., strength) and/or chemical property (e.g., hydrophilicity/hydrophobicity) of the material in which the structure is formed. Additives can be dispersed throughout the structure precursor material and/or can be incorporated within certain region(s) of a structure. In some cases, additives can be incorporated during formation of the structure by a three-dimensional fabrication process; in other cases, additives can be incorporated into the structure after the overall shape of the structure has been formed. Additives can also be incorporated into and/or onto a structure by adsorption or by chemically reacting the additive onto the surface of the polymer, i.e., by coating or printing the additive onto the structure. Non-limiting examples of additives include bioactive agents (e.g., therapeutic agents, proteins and peptides, nucleic acids, polysaccharides, nucleic acids, and lipids, including anti-inflammatory compounds, antimicrobial compounds, anti-cancer compounds, antivirals, hormones, antioxidants, channel blockers, and vaccines), surfactants, imaging agents, and particles. If desired, additives may be processed into particles using spray drying, atomization, grinding, or other standard techniques. In some cases, additives can be formed into emulsifications, micro- or nano-particles, liposomes, or other particles that can be incorporated into the material of a structure. In some embodiments, composite structures for tissue engineering and/or organ replacement can be formed by combining inorganic and organic components. Particles incorporating an additive can have various sizes; for example, particles may have a cross-sectional dimension of less than 1 mm, less than 100 microns, less than 50 microns, less than 30 microns, less than 10 microns, less than 5 microns, less than 1 micron, less than 100 nanometers, or less than 10 nanometers.

In some cases, it is desirable to release an additive from portions of a structure when the structure is in its environment of use (e.g., implanted in a mammalian body). Release of an additive may include hydrolysis and/or degradation of the polymer forming the structure. The release rate of the additive can be determined, in some instances, by the degradation rate of the polymer. The release rate of the additive can be controlled by the distribution of the additive throughout the polymer and/or by variation of the polymer microstructure (e.g., density of the polymer) such that the degradation rate varies with certain portions of the structure.

A structure precursor material described herein may have any suitable viscosity to make it compatible with a three-dimensional fabrication technique. In some embodiments, the viscosity of the precursor structure material is between 1-1,000, centipoise (cps), between 1,000-2,000 cps, between 2,000-5,000 cps, between 5,000-10,000 cps, between 10,000-15,000 cps, between 15,000-20,000 cps, between 20,000-25,000 cps, between 25,000-30,000 cps, between 30,000-35,000 cps, between 35,000-40,000 cps, between 40,000-45,000 cps, or between 45,000-50,000 cps. In certain embodiments, the viscosity of the precursor structure material is greater than 10,000 cps, greater than 20,000 cps, greater than 30,000 cps, greater than 40,000 cps, greater than 50,000 cps, or greater than 60,000 cps. Viscosity of the structure precursor material may be decreased by various means such as my adding a diluent to the material and/or increasing the temperature of the material. The viscosity of the material may be increased by various means such as by adding a filler (e.g., particles) or a viscous fluid to the material and/or decreasing the temperature of the material.

A component of a structure precursor material described herein may have any suitable molecular weight. In some cases, a component has a molecular weight between 10-100 g/mol, between 100-1,000 g/mol, between 1,000-5,000 g/mol, between 5,000-10,000 g/mol, between 10,000-15,000 g/mol, between 15,000-20,000 g/mol, between 20,000-25,000 g/mol, between 25,000-30,000 g/mol, between 30,000-35,000 g/mol, between 35,000-40,000 g/mol, between 40,000-45,000 g/mol, or between 45,000-50,000 g/mol. Components having a molecular greater than 50,000 g/mol can also be used.

Any suitable molecular weight ratio of polymerizable/crosslinkable and precipitating components can be used in structure precursor materials described herein. For example, the molecular weight ratio of a first, polymerizable and/or crosslinkable component to a second, precipitating component greater than or equal to 0.01:1, greater than or equal to 0.05:1, greater than or equal to 0.1:1, greater than or equal to 0.2:1, greater than or equal to 0.4:1, greater than or equal to 0.6:1, greater than or equal to 0.8:1, greater than or equal to 1:1, greater than or equal to 1.2:1, greater than or equal to 1.5:1, greater than or equal to 2:1, greater than or equal to 3:1, greater than or equal to 5:1, greater than or equal to 10:1, or greater than or equal to 20:1.

As described herein, in some embodiments, structure precursor materials are designed to have certain weight ratios of a first component and a second component. The first component may be a material that can be polymerized and/or crosslinked and the second component may be a material that can precipitate upon exposure to a precipitation medium. The ratio of the two components can change the physical properties (e.g., hardness) of the final porous structure. Additionally, in some cases the pore size of the final porous structure can be varied by changing the ratio of the components. For instance, in some embodiments, structure precursor materials including an increasing concentration of a polysulfone precipitating component relative to a FullCure™ polymerizable/crosslinkable component can result in smaller pore sizes. Accordingly, the weight ratio of a precipitating component to a polymerizable/crosslinkable component in a structure precursor material may vary depending on the desired pore size in the final porous structure, and may be, for example, greater than or equal to 0.2:1, greater than or equal to 0.4:1, greater than or equal to 0.6:1, greater than or equal to 0.8:1, greater than or equal to 1:1, greater than or equal to 1.2:1, greater than or equal to 1.5:1, greater than or equal to 2:1, greater than or equal to 3:1, greater than or equal to 5:1, greater than or equal to 8:1, greater than or equal to 10:1, greater than or equal to 15:1, or greater than or equal to 20:1.

The “pore size” of a structure refers to the length of the shortest line (e.g., cross-sectional dimension) parallel to a surface of the structure connecting two points around the circumference of a pore and passing through the geometric center of the pore opening. Pore sizes may be determined using techniques such as visible light microscopy, scanning electron microscopy (SEM), and filtration methods, as described in more detail below.

The cross-sectional shape (circular, oval, triangular, irregular, square or rectangular, or the like), number, and dimensions of the pores can be varied to suit a particular application. In one particular embodiment, the pores have an essentially circular cross-sectional profile. In some cases, the pores may have a smallest diameter that is smaller than a smallest cross-sectional dimension of a species to which the structure may be exposed. These pores may, for example, prevent passage of the species across the pore, e.g., from a first side to a second side of a porous structure. In other cases, the pore size may be selected to be much larger than a species to which the structure may be exposed. Furthermore, in some instances, the spatial distribution of the pores may be controlled.

In addition to the methods described above regarding fabrication of pores by removal of a component from a structure precursor material, other methods of creating pores in a structure can also be used. In some embodiments, more than one technique for introducing porosity in a structure can be used. For instance, porosity can be induced in a structure by methods such as, for example, phase inversion, solution casting, emulsion casting, and polymer blending. For instance, pores can be fabricated directly by a three-dimensional fabrication technique used to fabricate the structure. E.g., arrays of holes or pores can be drawn onto a scanned image to form a porous skeleton of the imaged tissue or organ. In other words, the pores can be fabricated using the same fabrication technique used to form the structure. In some cases, pores can be designed and printed with an offset. Additionally and/or alternatively, if desired, a porous material can be used to coat a surface of the structure. The porous material may include, for instance, more than one component having different solubility in certain solvents. For example, a first component may include the polymer in which the structure is formed, and a second component may include particles that are not soluble in the polymer, but which can be subsequently dissolved in a solvent that dissolves the particles. After the structure is coated with the porous material, the structure can be soaked in a solvent that dissolves the second component, e.g., to leach out the second component from the porous material.

Accordingly, structures described herein may comprise pores having a wide range of pore sizes. The pores of a structure may be uniform in size, or may vary in size if desired. In some embodiments, structures described herein are constructed to have a relatively homogeneous pore size distribution, for example, such that no more than about 5% of all pores deviate in size from the average pore size by more than about 20%, in some cases, by no more than about 10%, and in other cases, by no more than about 5%. The pore size of the structure may be less than or equal to 1 mm, less than or equal to 100 microns, less than or equal to 50 microns, less than or equal to 40 microns, less than or equal to 30 microns, less than or equal to 10 microns, less than or equal to 5 microns, less than or equal to 1 micron, or less than or equal to 100 nm. In embodiments including more than one pore size, a combination of pore sizes such as those described above, can be included in a structure.

As described herein, certain porous structures described herein may have sharp molecular weight cutoffs (MWCO). For example, at least 95% of the pores of a structure may have a MWCO of less than or equal to 5 kD, less than or equal to 10 kD, less than or equal to 15 kD, less than or equal to 20 kD, less than or equal to 25 kD, less than or equal to 30 kD, greater less or equal to 40 kD, less than or equal to 45 kD, less than or equal to 50 kD, less than or equal to 55 kD, less than or equal to 60 kD, less than or equal to 65 kD, less than or equal to 70 kD, less than or equal to 75 kD, less than or equal to 80 kD, or less than or equal to 100 kD.

Certain porous structures described herein may be able to exclude components having various sizes. For example, at least 95% of the pores of a structure may be able to exclude components having a size of greater than or equal to 1 mm, greater than or equal to 100 microns, greater than or equal to 50 microns, greater than or equal to 40 microns, greater than or equal to 30 microns, greater than or equal to 10 microns, greater than or equal to 5 microns, greater than or equal to 1 micron, or greater than or equal to 100 nm.

In some embodiments, porous polymeric structures can be prepared in-situ with inherent properties that are suitable for use as biohybrid organs scaffolding. In some cases, structure precursor materials can be casted as flat sheet separation membranes and/or hollow fibers. The materials may have permeation properties ranging from the ultrafiltration to microfiltration ranges. These properties can allow the membranes to separate substances having different molecular weights. In certain embodiments, the membranes can serve as bioactive membranes without further processing (e.g., further modification of the membrane surface), for example, the membranes may show good biocompatibility and cell adherence without extracellular matrix (ECM) surface coatings.

In certain embodiments, structures such as flat membranes or hollow fibers can be fabricated using polysulfone (PS, a precipitating component) and Fullcure™ 700 monomer (FC, a crosslinkable component). The membranes and/or hollow fibers can be prepared with a controllable MWCO of between, for example, 5-100 kDa, which may allow the transport of certain ions, nutrients, waste products, protein-bound toxins, etc. The structures may be functionalized and modified by wet and dry surface chemistry. In some cases, biospecific ligands can be covalently bound or adsorbed to the surfaces to support the attachment and function of kidney epithelial cells on one side of the structure, and to achieve a good hemocompatibility on the blood-contacting side of the structure. If both properties cannot be combined in one structure, replacement can be achieved by the application of a specific fiber-in-fiber design for hollow fibers.

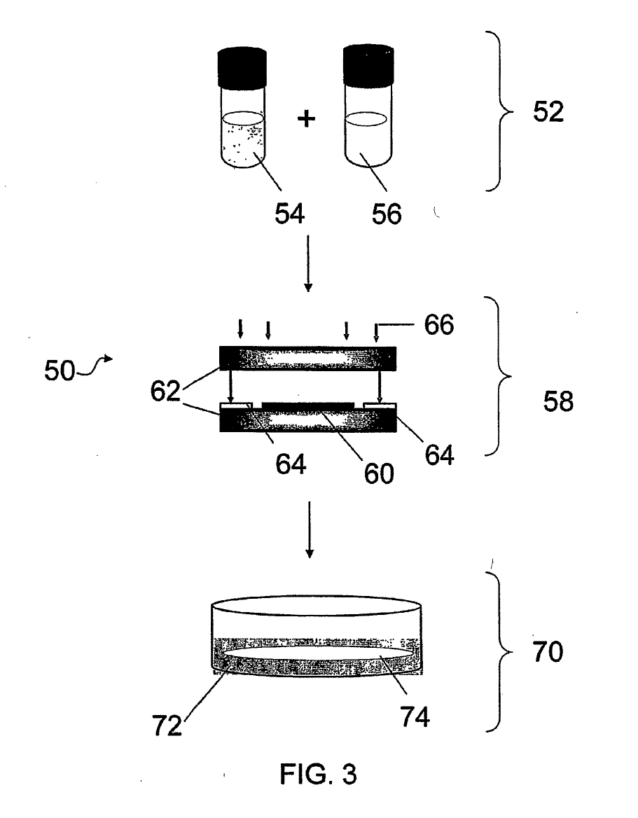

FIG. 3 shown an example of a process for fabricating porous structures in the form of membranes. As shown in the embodiment illustrated in FIG. 3, first component 54 (e.g., a solution of polyethersulfone) including a solvent (e.g., dimethylacetamide) and second component 56 (e.g., Fullcure™ 700 monomer) may be mixed to form a structure precursor material. The structure precursor material 60 may be poured and sandwiched between two glass plates 62 separated by one or more spacers 64 that controls the membrane thickness. The whole assembly can then be subjected to UV radiation, as indicated by arrows 66, which can cause polymerization and/or crosslinking of one component of the structure precursor material (e.g., Fullcure™ 700). The structure precursor material may be removed from the assembly and then subjected to a phase inversion process 70, whereby the structure precursor material is placed in a precipitation medium 72 (e.g., water) to allow precipitation of one component of the structure precursor material (e.g., polyethersulfone), and/or removal of a component of the material (e.g., the solvent), to generate porous structure 74. The membranes can be highly permeable and may have attractive biofunctional characteristics (such as sharp MWCO), and may possess high filtration and diffusion fluxes, and have good mechanical strength, biocompatibility and formability. Such structures may be used in applications involving, for example, waste water treatment and/or purification.

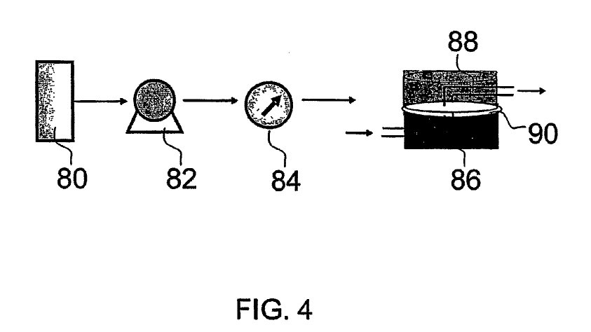

In some instances, pore sizes and/or the MWCO of a membrane can be measured by a filtration setup, as shown in FIG. 4. As illustrated in the embodiments of FIG. 4, reservoir 80 can pump a feed solution from lower compartment 86 to upper compartment 88 through porous membrane 90. Permeate may be collected in the upper compartment. The pumping and pressure of the solution can be controlled by pumping system 82 and pressure system 84, respectively. Using such a system, the flux of the solution through the membranes can be measured under steady-state flow. The passing of different solutions containing solutes of known molecular weights (and/or sizes) can cause changes in the flux through the membrane, and the different concentrations of feed solution and permeate solution can be used to determined the pore size and/or molecular weight cutoff of the membrane, as described in more detail in the Examples.

In some embodiments, articles of the invention can be used as biocompatible structures for tissue engineering and/or organ replacement. Such structures may be formed, for example, by three-dimensional fabrication techniques. In some embodiments, the biocompatible structures are scaffolds for cells that can be used as tissue engineering templates and/or as artificial organs. The structures may be three-dimensional and can mimic the shapes and dimensions of tissues and/or organs, including the microarchitecture and porosities of the tissues and organs. For instance, certain embodiments of the invention can be fabricated to include very small features (e.g., less than 20 microns), such as small pore sizes, small cavities, and/or structures having thin walls. These features are particularly well-suited for structures involving hollow and epithelial organs. In some cases, a structure formed by three-dimensional fabrication comprises a wall defining a cavity and a plurality of pores in at least a portion of the wall. The pores may permeate the wall, at least at selected portions of the wall or all throughout the wall, and enable exchange of a component (e.g., a molecule and/or a cell) between a portion interior to the cavity and a portion exterior to the cavity. For instance, pores may allow delivery of molecules, cell migration, and/or generation of connective tissue between the structure and its host environment. Advantageously, structures including pores of uniform size can be fabricated by methods described herein. For example, pores may have an average pore size of less than or equal to 20 microns, wherein no more than about 5% of all pores deviate in size from the average pore size of the plurality of pores by more than about 20%. Structures of the invention can be implanted into a mammal, or alternatively and/or additionally, can be used ex vivo as bioartificial assist devices.

In some cases, structures can be fabricated to include substructures. For instance, a large vessel may be fabricated to include small vessels within the large vessel. Surfaces of substructures may also be modified, i.e., in a fashion described above. For example, in one embodiment, a wall of the large vessel may be modified with a first growth factor to induce growth of a first type of cell on the wall of the large vessel, and a wall of the small vessel may be modified with a second growth factor to induce growth of a second type of cell on the wall of the small vessel. Substructures may include pores that allow exchange of a component between an interior cavity portion of the substructure and a portion exterior to the substructure, i.e., between a cavity portion of the substructure and a cavity portion of a larger structure.

A wide variety of artificial tissues and organs can be fabricated as three-dimensional structures using methods described herein. In some embodiments, the structures can be used as templates for cell growth, which may be applied towards tissue engineering and/or organ replacement. For structures to be used in vivo, cells and/or tissues may be grown on a structure prior to the structure being implanted, or alternatively, the structure may be positioned directly into a mammalian system where the body's cells naturally infiltrate the structure.

In some particular embodiments, structures may be formed in the shape of organs that include a cavity portion. For instance, structures including a cavity portion may include hollow organs and/or epithelial organs such as vessels, lung, liver, kidney, pancreas, gut, bladder, and ureter, as described in more detail below. A cavity of a structure, as used herein, refers to a substantially enclosed space defined by a wall of the structure, in which a plane can be positioned to intersect at least one point within the cavity and the structure, where it intersects the plane, completely surrounds that point. The cavity and can be closed or open. For example, in one embodiment, a cavity may be defined by the interior space within a tube of a blood vessel. In another embodiment, a cavity may be defined by the hollow space inside a bladder. As such, cavities may have a variety of shapes and sizes. A space within a cavity is referred to as an interior cavity portion, and a space outside of the cavity is referred to as a portion exterior to the cavity. The cavity may be filled with fluid, air, or other components. In some cases, a cavity may be lined with one or more layers of cells or tissues. The layers of cells or tissues may form, for instance, membranes or walls of the tissue or organ. In some instances, the lining of a cavity can comprise pores that allow exchange of a component between a portion interior to the cavity and a portion exterior to the cavity, as described in more detail below.

A cavity of a structure may vary in volume and may depend, in some instances, on the tissue or organ in which the structure mimics. The volume of the cavity may be, for instance, less than 1 L, less than 500 mL, less than 100 mL, less than 10 mL, less than 1 mL, less than 100 microliters, less than 10 microliters, less than 1 microliter, less than 100 nanoliters, or less than 10 nanoliters, where volume is measured as within that portion of the structure that is enclosed.

A wall of a structure defining a cavity portion can vary in thickness, and may also depend on the tissue or organ in which the structure mimics. In some cases, thick walls (e.g., greater than 500 microns thick) may be suitable for certain structures (e.g., a bladder) that may, for example, require slow or relatively little exchange of components between portions interior and portions exterior to the cavity. Thin walls (e.g., less than 50 microns thick) may be applicable to some structures (e.g., alveoli) that may, for example, require quick exchange of components between portions interior and portions exterior to the cavity. In certain embodiments, a wall of a structure can be less than 1 mm thick, less than 500 microns thick, less than 200 microns thick, less than 100 microns thick, less than 50 microns thick, less than 30 microns thick, less than 10 microns thick, less than 5 microns thick, or less than 1 micron thick.

In some instances, a cavity may be defined by an inner diameter of a certain distance. “Inner diameter”, as used herein, means the distance between any two opposed points of a surface, or surfaces, of a cavity. For example, the inner diameter of a blood vessel may be defined by the distance between two opposing points of the inner wall of the vessel. Inner diameters may also be used to describe non-spherical and non-tubular cavities. A cavity may have an inner diameter of, for example, less than 10 cm, less than 1 cm, less than 1 mm, less than 500 microns, less than 200 microns, less than 100 microns, less than 50 microns, less than 30 microns, less than 10 microns, less than 5 microns, or less than 1 micron.

In some embodiments, a structure may include a cavity having more than one portion, for example, a first and a second cavity portion may be interconnected, which allows a substance to pass freely between the cavity portions. Additionally or alternatively, the structure may include more than one cavities (e.g., in a case where the cavities are not interconnected). For instance, in one embodiment, a cavity of a structure may include at least a first and a second portion, the first portion of the cavity being defined by a first inner diameter and the second portion being defined by a second inner diameter. In another embodiment, a structure may include a first cavity having a first inner diameter and a second cavity having a second inner diameter. The second cavity may be defined, for instance, by that of a substructure. For the above cases, the first and second inner diameters may be different; for example, the ratio of the first inner diameter to the second inner diameter can be greater than 1:1, greater than 2:1, greater than 5:1, greater than 10:1, greater than 20:1, greater than 50:1, greater than 100:1, greater than 200:1, or greater than 500:1. Some structures, such as certain vessels, may have a first cavity portion having the same inner diameter as that of a second cavity portion, i.e., the ratio of the inner diameters of the first and second portions may be 1:1. Additional examples of such structures are described in more detail below.

In mimicking tissues and/or organs of the body, different types of cells can be arranged proximate a structure in sophisticated micro-architectures that are responsible for the complex functions of the tissue or organ. Thus, microstructures having dimensions and arrangements closely related to the natural conditions of the tissue or organ can be formed. The design of the structure and the arrangement of cells within the structure can allow functional interplay between relevant cells, e.g., between cells cultured on the structure and those of the host environment. These factors may also enable appropriate host responses, e.g., lack of blood clotting, resistance to bacterial colonization, and normal healing, when implanted into a mammalian system.

The present inventors have realized the importance of addressing geometry, size, mechanical properties, and bioresponses in fabricating structures for tissue engineering and organ replacement, especially for structures involving hollow and epithelial organs, as described in more detail below.

In one aspect of the invention, tissues and organs of interest include those of the circulatory system. The circulatory system includes the heart (coronary circulation), the blood vessel system (systemic circulation), and the lungs (pulmonary circulation). The circulatory system functions to deliver oxygen, nutrient molecules, and hormones to the body, and to remove carbon dioxide, ammonia and other metabolic waste from parts of the body.

Coronary circulation refers to the movement of blood through the tissues of the heart. In some cases, portions of the heart become diseased. For instance, heart tissue may not receive a normal supply of food and/or oxygen, or certain structures forming the heart, such as heart valves, may not be operating normally. In the latter case, when heart valves are functioning properly, the flaps (also called leaflets or cusps) of the valves open and close fully. Proper function of heart valves may cease when the valves do not open enough or do not let enough blood flow through; this condition is called stenosis. When the valves do not close properly, blood may leak into places where it shouldn't; this condition is called incompetence or regurgitation. In these instances, heart valves may need to be replaced. In one embodiment, methods described herein can be used to fabricate heart valves (e.g., tricuspid, pulmonary, mitral, and/or aortic valves) that are coated with films of additives known to prevent blood clotting. In another embodiment, an artificial valve may incorporate additives such as antibiotics, which can prevent endocarditis, an infection of the heart's lining or valves. In some cases, an artificial valve may comprise a combination of additives, such as the ones mentioned above. The heart valves can be used in vivo to replace diseased heart valves, and/or in vitro as a scaffold template for cell seeding.

In another embodiment, three-dimensional fabrication techniques can be used to form structures of the blood vessel system, including arteries, veins, capillaries, and lymphatic vessels. The blood vessel system keeps blood moving around the body inside the circulatory system.

Arteries carry blood that is full of oxygen from the heart to all parts of the body. As the arteries get further away from the heart, they get smaller. Eventually, arteries turn into capillaries, the smallest blood vessels, which go right into the tissues. Here, the blood in the capillaries gives oxygen to the cells and picks up the waste gas, carbon dioxide, from the cells. The capillaries are connected to the venules, the smallest veins in the body, and the veins get bigger as they carry the blood back towards the heart. The capillaries are the points of exchange between the blood and surrounding tissues. Components can cross in and out of the capillaries, for instance, by passing through or between the cells that line the capillary.

Structures for use as templates for cell growth can be designed to mimic a variety structures of the blood vessel system. In some embodiments, structures can serve as templates for triggering controlled in-growth of vascular structures or complete artificial vessel replacements. Such structures may be used for the induction of vessels in vivo.

Structures described herein may be formed in the shape of a tube including interior cavity portion and a portion exterior to the cavity. The structure may have a first end portion and a second end portion, which may be opened or closed. In some cases, the end portions and may be used to connect the structure to ducts of a patient. The dimensions of the structure may vary depending on the particular body part the structure will mimic, where the structure will be positioned in the body, the size of the patient, etc. For example, the structure may have an inner diameter and/or outer diameter of less than 10 mm, less than 5 mm, less than 2.5 mm, less than 1.5 mm, or less than 1 mm. In some embodiments, the structure can be transplanted into a mammalian body and may have a length between 10 mm and 100 mm, or between 25 mm and 75 mm (e.g., 50 mm); the inner diameter may have a length of about 0.5 mm, and the outer diameter may have a length of about 1.5 mm. The thickness of the wall of the structure may be defined by the difference between inner and outer diameters. Thicknesses of the wall can range from a few microns (i.e., a few cells) to millimeters thick.

In some cases, the structure can have a plurality of pores in at least a portion of the structure. The pores can vary in size; for instance, large pores (e.g., greater than 100 microns) may be suitable for growing large vessels through the pores, and/or for facilitating high exchange of components between an interior cavity portion and portion exterior to cavity. Small pores (e.g., less than 100 microns) may be suitable for growing small vessels through the pores, and/or for facilitating relatively low exchange of components across the wall of the structure. Such structures may be implanted into a mammal, or used in vitro.

In some cases, structures may include one or more additional substructures. For instance, a tubule may be fabricated to include a substructure such as a vessel. The vessel may be positioned in at least a portion within an interior cavity portion of the tubule, or it may be positioned exterior to the cavity. In some cases, the vessel may pass across a pore of the tubule, or the vessel may be interwoven between pores of the tubule. As such, the tubule may include at least a first cavity (e.g., an interior cavity portion of the tubule) and a second cavity (e.g., a cavity portion of the vessel). The ratio of the inner diameter of the first cavity to the inner diameter of the second cavity may be, for example, greater than 1:1, greater than 2:1, greater than 5:1, greater than 10:1, greater than 20:1, greater than 50:1, greater than 100:1, greater than 200:1, or greater than 500:1.

In some embodiments, structures described herein can be used to replace a section of a blood vessel in a patient. Such a structure may include an interior cavity portion having an inner diameter, a portion exterior to the cavity, a first end, and a second end. The structure can also include sections that can be used as interconnecting lumens for connecting the structure to one or more ducts of a patient. If desired, the structure can be designed to include a plurality of such sections. The sections may each be defined by cavity portions having a certain inner diameter. In some cases, the ratio of the inner diameter of a first cavity portion to the inner diameter of a second cavity portion can be equal to 1:1, greater than 1:1, greater than 2:1, greater than 5:1, greater than 10:1, greater than 20:1, greater than 50:1, or greater than 100:1.

The wall of the structure may have a thickness of less than 5 mm, less than 1 mm (e.g., 0.5 mm), or less than 0.5 mm. In one particular embodiment, a wall of a structure has a thickness of 0.5 mm. In some cases, the wall may be formed in an elastic material that allows stretching, recoiling, and/or absorption of pressure in response to, for example, pumping of the heart and fluid flow through the structure. If desired, before implanting the structure into a patient, smooth muscle cells may be grown onto all, or portions, of the wall of the structure. These muscle cells may contract and expand to control the diameter, and thus the rate of blood flow, through the structure (e.g., contraction and expansion of muscle cell may cause the structure to dilate and constrict, respectively). In some cases, an additional outer layer of connective tissue may be grown onto the structure. A layer of elastic fibers may also be grown onto the structure to give it greater elasticity, if desired. In some embodiments, the structure can be made from a biodegradable polymer that degrades, for example, after healthy tissues have re-grown and have integrated into the body.

In some embodiments, structures formed by methods described herein are designed to mimic capillaries, which can allow exchange of components such as nutrients, wastes, hormones, and white blood cells, between the blood and surrounding environment. The surrounding environment may include, for example, the interstitial fluid and/or surrounding tissues. The artificial structure may include a cavity portion comprising a wall having a thickness of, for example, 0.5 mm or any other suitable thickness, which can be lined with endothelial cells. In some cases, a wall of the capillary has a thickness of a single cell. In one embodiment, capillary structures may include small pores or holes that may be less than 50 microns, less than 10 microns (e.g., about 1 micron) in size between the cells of the capillary wall, allowing certain components to pass in and out of capillaries, e.g., between an interior cavity portion and a portion exterior to the cavity (e.g., the surrounding tissues). The pores may allow certain small components such as certain dissolved molecules (e.g., small ions) to pass across the pores, but may inhibit larger components such as proteins from passing across. In another embodiment, exchange of components across a capillary wall can occur by vesicles in the cells of the capillary wall that pick up components from the blood (e.g., in the interior cavity portion of the capillary), transport them across the capillary walls, and expel them into the surrounding tissue (e.g., into a portion exterior to the cavity of the capillary). In yet other embodiment, components may exchange between an interior cavity portion and a portion exterior to the cavity via passage through the cell lining. For instance, components may diffuse from the blood into the cells of the capillary walls, and then into the surrounding tissue. Artificial capillaries may also be designed to include one or more branching structures, which can create a greater surface area through which the exchange of components can occur.

In another aspect of the invention, structures are fabricated to mimic tissues and/or organs of the digestive track. The digestive tract encompasses the oral cavity, esophagus, stomach, small and large intestines, rectum, and anus. The different parts of the digestive tract may display a similar histo-architecture, i.e., each part may comprise a muscle wall that is covered by the mucosa, which contains epithelial cells. These organs can be affected by diseases such as cancer, infection, etc. Diseased organs of the digestive track typically require operations that include resections of the diseased segment. These removed segments can be replaced with artificial structures of the present invention. In some embodiments, structures can be fabricated to mimic a diseased section. The structure may be used as a scaffold for the in-growing of natural mucosa from healthy cells of a patient. This scaffold can then be implanted into the patient. In one embodiment, this approach is applied to so-called gut pouches to replace the continence function of the gut. Like artificial structures of the circulatory system, structures of the digestive track can be formed in biodegradable polymers.

In another aspect of the invention, structures are fabricated to mimic gut-associated glands. Gut-associated glands include the salivary glands, the liver, and the pancreas. All three organs are made up of specialized epithelial cells with endocrine and exocrine functions. In one embodiment, structures can be fabricated to mimic portions of the liver. The liver is comprised mainly of lobules containing hepatocytes that are arranged in plates. In between the hepatocyte plates, blood-containing sinusoids can be found. The center of the lobule is the central vein, and this vessel receives blood from the sinusoids. In some embodiments, artificial structures in the shape of liver lobules can be fabricated. The structure may include a scaffold for plating and growing hepatocytes. The scaffold can be designed with a specific micro-architecture that allows spatial control of the seeding of cells. The structure may also include sinusoidal structures, which can function as cavities for containing blood. The plates can be filled with hepatocytes in the inner space of the scaffold, and a plate wall adjacent a center can be coated with endothelial cells. In certain embodiments, the liver lobules can have dimensions of approximately 0.7 mm×2 mm. The structure can be fabricated to have pores that can facilitate exchange of a component. For example, exchange of a component may occur via pores between the blood contained in the sinusoidal structures (e.g., an interior cavity portion) and the hepatocytes, which may be located at a portion exterior to the cavity. Pores can be fabricated to have a variety of sizes. Generally, for liver lobules, pores may be fabricated to have a cross-sectional dimension in the micron range.

In another embodiment, structures for tissue engineering and/or organ replacement can be fabricated to mimic portions of the pancreas. The pancreas is a mixed exocrine-endocrine gland that produces hormones such as insulin and glucagons, as well as pancreatic enzymes that help digest acids and macromolecular nutrients (e.g., proteins, fats and starch). The hormone-producing cells are aggregated in the islets of Langerhans. Pancreatic islets are scattered throughout the pancreas. Like all endocrine glands, pancreatic islets secrete their hormones into the bloodstream and not into tubes or ducts. Because of the need to secrete their hormones into the blood stream, pancreatic islets are surrounded by small blood vessels (e.g., capillaries). The islets are also highly vascularized, facilitating the exchange of hormones between the islets and the vessel system. In certain embodiments of the invention, structures in the shape of island-like structures are fabricated using techniques described herein. The artificial island-like structures can be designed to have a specific micro-architecture that can enable endocrine cells to be seeded in preformed locations, i.e., near structures that are designed to guide the capillaries. Like the structures described above, structures that mimic portions of the pancreas can be formed in biodegradable polymers if desired. These artificial pancreatic structures may be used to treat diseases such as diabetes mellitus.

In another aspect of the invention, structures are fabricated to mimic endocrine organs. The endocrine organs include the adrenals, thyroid, parathyroid, and pineal gland. These organs are made up of endocrine (i.e., hormone-producing) cells that are located very close to the capillaries, as described above for the islets of Langerhans. The close proximity of these organs to the capillaries allows the blood circulating factors to leave the capillaries and become bound to cell receptors on the endocrine cells, triggering the release of hormones. The released hormones diffuse into the capillaries, and are subsequently distributed in the body to bind with receptors in other tissues. In some embodiments, endocrine structures can be fabricated to have a specific micro-architecture that allows the seeding of cells within certain locations of the structure. Artificial endocrine organs may be fabricated to have a high degree of vascularization that facilitates the exchange of components between the organ and the capillaries. In some cases, artificial endocrine organs are made with high porosity. The pores may have a variety of sizes depending on the particular organ. Like the structures described above, structures that mimic endocrine organs can be formed in biodegradable polymers if desired. Artificial endocrine organs may be applied, for instance, towards treating insufficient production of hormones in glands.

In another aspect of the invention, structures are fabricated to mimic portions of the respiratory system. The respiratory system includes the trachea and the lungs. In one embodiment, a structure can be fabricated to replace diseased or damaged portions of the trachea. The trachea is a cartilaginous and membranous-ringed tube where air passes to the lungs from the nose and mouth. The trachea bifurcates into right and left mainstem bronchi. Artificial trachea may be fabricated to include similar architecture and mechanical properties to that of healthy trachea. For instance, the artificial structure may include ring-like portions made from an elastic polymer that resembles cartilage. In some cases, cartilage cells (e.g., hyaline cartilage) from healthy trachea can be seeded and grown into the artificial structure. The artificial structures can be lined with ciliated cells, used to remove foreign matter (e.g., dust) from the airway so that they stay out of the lungs.

In one embodiment, a structure can be fabricated to replace diseased or damaged portions of the lung. The lungs include air-conducting segments such as the bronchioles, numerous small tubes that branch from each bronchus (a branch of the trachea) into the lungs. The lungs also include the alveoli, the respiratory portions where gas exchange takes place. The air-conducting portions include a wall that is lined by respiratory epithelium, which is responsible for producing mucous fluid. In some cases, structures are fabricated to mimic portions of the air-conducting segments. For instance, artificial bronchioles may be fabricated to have a thickness of less than 10 mm, less than 1.0 mm (e.g., 0.5 mm), or less than 0.5 mm, and a diameter of less than about 10 mm, less than about 5 mm (e.g., 2 mm), or less than about 2 mm. The thickness and diameter of the bronchiolar structure will depend, of course, on the position of the structure within the lung, the size of the patient, etc. All structures of the air-conducting portion can be formed as the artificial interposed segments or as templates for engineered tissue constructs. For instance, in some cases, the artificial structure may form a scaffold for growing connective tissue and smooth muscle cells within the walls of the structure. The walls may also be lined with epithelial cells, which can comprise three types of cells: ciliated cells, non-ciliated cells, and basal cells. In some particular embodiments, certain artificial structures, such as those that mimic terminal bronchioles, can be fabricated to include artificial alveoli in the walls of the structure.

In some embodiments, structures are fabricated to mimic alveoli. Alveoli are small, thin-walled air sacs (i.e., cavities) at the end of the bronchiole branches having cross-sectional dimensions on the order of 200 microns. Proximate the alveolar walls are pulmonary capillaries where gas exchange occurs between blood in the capillaries and inhaled air in the alveoli. For instance, to reach the blood, oxygen diffuses through the alveolar epithelium, a thin interstitial space, and the capillary endothelium; carbon dioxide follows the reverse course to reach the alveoli. In certain embodiments of the invention, artificial alveolar structures can be fabricated with natural dimensions and with porous walls for gas exchange. Pores in the walls of the alveoli may allow exchange of a component (e.g., a gas) between an interior portion of the alveoli (e.g., an interior cavity portion) and the interstitial space surrounding the alveoli (e.g., a portion exterior to the cavity portion). Artificial alveolar structures may be formed in an elastic material that gives the alveoli mechanical stability while allowing expansion and contraction of the structures. In some cases, the artificial alveolar structures may form scaffolds for growing cells, i.e., the structures may be lined with epithelial cells such as Type 1 and Type 2 pneumocytes. Artificial alveoli can be used to help increase the oxygen content in patients with respiratory deficiencies.

In another aspect of the invention, structures are fabricated to mimic portions of urinary system. The urinary system comprises the kidneys, ureters, the urinary bladder, and the urethra. In some cases, a structure can be fabricated to replace diseased or damaged portions of the kidney. The kidney is formed from a plurality of nephrons, which include the glomerulus and the proximal and distal convoluted tubules. The glomerulus represent the filtration stations, which contain tuffs of capillaries where the ultrafiltrate is pressed out. In some embodiments, a structure can be fabricated in the form of a porous looped superstructure. In one embodiment, the structure can be used as an artificial glomerulus. In another embodiment, the structure can be used as artificial proximal and/or distal convoluted tubules. In some cases, the structure can include a plurality of loops, which can be of the same or different dimensions. The structure can include at least one wall defining a cavity (e.g., a tubular portion). The cavity can have the same inner diameter throughout the structure, e.g., of about 40-500 microns in one embodiment, or between 50-100 microns in another embodiment. Alternatively, a first portion of the cavity may have an inner diameter different than that of a second portion of the cavity. The thickness of the wall can range, for example, from about 1-500 microns (e.g., 2-500 microns), 1-100 microns, or 2-100 microns. The wall may optionally include a plurality of pores that enable exchange of a component (e.g., water and ions) between a portion interior to the cavity and a portion exterior to the cavity. The pores may allow certain components to pass between interior and exterior portions of the cavity, e.g., based on size, charge, etc. In some cases, all, or portions, of the wall can be covered with films of nanometer to micron thickness. These films can form selective permeable membranes allowing certain components to pass between interior and exterior portions of the cavity. The structure may be used to process ultrafiltrate in such a way that the good substances (e.g., glucose and amino acids) become reabsorbed, and the wastes (e.g., urea) get discarded as urine. In certain embodiments, the structure can act as a hemofiltration system. Accordingly, the structure can be used to replace and/or aid the filtration function of the kidney.

Some embodiments of the invention include the formation of a plurality of cavities within a structure. For example, in the embodiment, a structure can be formed in the shape of a block, and may include a wall that defines a plurality of cavities. Cavities within the structure may be separate in some embodiments, or they may be interconnected in other embodiments. Cavities within the structure may have the same or different geometry and/or dimensions. Structures having a plurality of cavities can be used, for instance, to improve the surface-to-volume ratio in a hemofiltration system, e.g., for higher rate of reabsorption of electrolytes such as glucose and other metabolic products. In some cases, such structures may be combined with other embodiments of the invention. For instance, such structures can be combined with one or more artificial glomeruli to replace the main renal function with an extracorporeal module. In other instances, such structures can be combined with one or more artificial glomeruli as an implantable device to replace the main renal function in a mammalian system.

In some cases, structures including a plurality of cavities may include one or more additional substructures. For instance, such a structure may be fabricated to include a substructure such as a vessel. The substructure may be positioned in at least a portion of a cavity of the structure, or the substructure may be positioned exterior to the cavity. In some cases, a substructure may be interwoven between more than one cavities of the structure. As such, the structure may include at least a first cavity and a second cavity (e.g., a cavity portion of the vessel). The ratio of the inner diameter of the first cavity to the inner diameter of the second cavity may be, for example, greater than 1:1, greater than 2:1, greater than 5:1, greater than 10:1, greater than 20:1, greater than 50:1, greater than 100:1, greater than 200:1, or greater than 500:1.

In some cases, an artificial structure can be fabricated to replace diseased or damaged portions of the ureter and/or bladder. The ureter and bladder are hollow organs that include a wall, lined by a transitional epithelium, defining a cavity portion. Sometimes, this epithelium can be affected by cancer. Typically, to treat such a disease, a surgical operation is necessary whereby portions of the gut are removed and used to replace the reservoir function of the bladder, or the conductive function of the ureters. In some cases, this procedure causes the urethra to be affected by infection, leading to urethra stenosis. To circumvent these complications, diseased portions of the ureter and/or bladder may be replaced using artificial structures of the invention. Artificial structures may also be used to replace portions of the ureter and/or bladder to treat conditions such as urinary incontinence.

Structures formed by three-dimensional fabrication techniques can be used to replace portions of the ureters or urethra, or, they may be employed as artificial urinary bladders. The structures may be used for tissue engineering and/or organ replacement, in vivo or ex vivo. In one embodiment, a structure to be used as an artificial bladder includes a main body portion, an inlet for connecting to the ureters, and an outlet for connecting to the urethra. The structure may include a wall defining a cavity portion of the main body portion (e.g., a first cavity portion), a cavity portion of the inlet (e.g., a second cavity portion), and a cavity portion of the outlet (e.g., a third cavity portion). The cavity portions may have inner diameters ranging from, for example, about 0.01-5 mm, or 0.01-2 mm. In some instances, one cavity portion may have an inner diameter that is different from the inner diameter of another cavity portion of the structure. For example, the ratio between inner diameters of the second cavity portion and the first cavity portion may be greater than 1:1, greater than 2:1, greater than 5:1, greater than 10:1, greater than 20:1, greater than 50:1, or greater than 100:1.

The wall of the structure to be used as an artificial bladder may have a thickness ranging from, for example, about 0.01-5 mm, or 0.01-2 mm, depending on the volume of liquid in the artificial bladder, and may be formed in a flexible material to allow expansion and contraction of the bladder. In some cases, the wall is lined with cells and/or tissues before implanting the structure into a patient. For instance, the structure may serve as a template for different layers of tissues that form the bladder, e.g., the mucosa, submucosa, and muscularis layers. The mucosa includes the transitional epithelium layer, which can serve as a selective barrier between the organ an environment exterior to the organ. Underneath the epithelium layer can include the basement membrane, a single layer of cells separating the epithelial layer from the submucous layer (lamina propria). The submucous layer includes connective tissue that is interlaced with the muscular coat. The submucous layer can contain blood vessels, nerves, and in some regions, glands; in some embodiments, the structure can include such micro-architectures. Muscle cells defining the muscular layer may be positioned underneath the submucous layer.

Structure described herein may be capable of or modified to permit the adhering of various species to the surface of the structure or to a material coating a surface of the structure. For example, cells and/or biological molecules such as proteins, and the like may become immobilized with respect to various portions of the structure, including, for example, areas along the side walls of the pores, areas between the pores on a surface of the structure, or areas on top of the pores.

Some structures described herein may comprise an adhesive material selected to preferentially attract and/or bind a particular species, such as a cell or other biological species that is attached to, immobilized with respect to, or otherwise associated with at least one side of a structure. In certain embodiments, the adhesive material is a cell adhesive material. The term “cell adhesive material” as used herein may refer to any chemical or biological material to which a cell may adhere. In certain embodiments, such a cell adhesive material is configured as a continuous layer attached to a surface of at least one side of a structure. Such a cell adhesive material layer may comprise, any of a wide variety of species known in the art to be capable of binding to, specifically or non-specifically, membranes of biological cells or components thereof, such as for example, collagen or mixtures of collagen with polysaccharide, antibodies, ligands to cell surface receptors, antigens, lectins, integrins, selectins, bacterial derived affinity molecules such as Protein A or Protein G, derivatives thereof, mixtures thereof, any of the above associated with a gel or other layer-forming material, such as collagen, gelatin, agarose, acrylamide, chitosan, cellulose, dextran, an alginate, a carrageenan, etc., and the like.

Surface properties of the structures can be modified by various techniques. In some cases, surfaces of a structure can be modified by coating and/or printing an additive proximate the structure. In other cases, additives can be incorporated into the material used to form the structure (e.g., embedded in the structure during fabrication), as described herein. Surfaces may be modified with additives such as proteins and/or other suitable surface-modifying substances. For example, collagen, fibronectin, an RGD peptide, and/or other extracellular matrix (ECM) proteins or growth factors can be coated onto the structure, e.g., to elicit an appropriate biological response from cells, including cell attachment, migration, proliferation, differentiation, and gene expression. Cells can then be seeded onto surfaces of this structure. In one embodiment, cell adhesion proteins can be incorporated into certain channels and/or pores of a structure to facilitate ingrowth of blood vessels in these channels and/or pores. In another embodiment, growth factors can be incorporated into the structure to induce optimal cell growth conditions that triggers healthy tissue formation within certain regions of the structure. In yet another embodiment, a structure may comprise an additive such as a cell adhesive material positioned on one surface of a side of the structure, such as an inner cavity of a structure. In such an embodiment, a first type of cells can adhere to the inner cavity of the structure when the structure is exposed to a medium containing cells. Optionally, an outer portion of the structure may preferentially attract and/or bind a second type of cell when the structure is exposed to a medium containing cells.

In some cases, it may be desirable to modify all or portions of a surface with a material that inhibits cell adhesion, such as a surfactant (e.g., polyethylene glycol and polypropylene oxide-polyethylene oxide block copolymers). For instance, areas of a structure where it is not desirable for cellular growth can be coated with such materials, e.g., to prevent excessive soft connective tissue ingrowth into the structure from the surrounding tissue. In some cases, modification of surface properties of the structure can be used to position cells at specific sites within the structure. In some embodiments, a combination of cell-adhering and cell-inhibiting substances can be incorporated into various portions of a structure to simultaneously facilitate and inhibit cell growth, respectively.

In some embodiments, a structure can be coated with a porous material (e.g., a polymer such as a gel) prior to being coated and/or printed with a surface-modifying substance. For instance, in one embodiment, a structure can be fabricated using three-dimensional fabrication or another suitable technique to form a bioartificial kidney. In some instances, the structure can be modified with a substance; for instance, the structure can be first coated with a porous polymer, and then with a surface-modifying substance such as collagen, which may be used to facilitate cell adhesion. Cells (e.g., vascular cells) can then be seeded into and/or onto the modified structure. In some cases, the structure may include another layer of cells (e.g., proximal tubule cells). The device may mimic the function of a kidney to allow flow of blood and ultra-filtrate in and out of the structure.

If desired, structures of the invention can be coated with a porous polymer. A porous polymer coating a structure can be used for a variety of purposes. For example, a porous polymer may be used to form small pores (e.g., having a cross sectional dimension on the order of 1-20 microns, or within the range of porosity of the polymer) within a larger pore (e.g., having a cross sectional dimension on the order of 20-200 microns) of the structure. In some cases, the porous polymer may allow sustained release of an active agent from the polymer, e.g., to facilitate cell growth and/or adhesion as a function of time. In other cases, the porous polymer can influence transport of components from a first to a second position of the structure. In yet other cases, a porous polymer coating a structure can reduce the surface roughness of the structure, as described below. One non-limiting example of a suitable porous polymer is polysulfone.

A variety of techniques, such as those described below, can be used to fabricate or shape structures described herein. After or during the process of carrying out such techniques, the structure may be exposed to a precipitation medium to form cell growth template structures having a network uniform pores.

In some embodiments, structures described herein are fabricated at least in part by using one or more ejection processes, such as jetting processes, including thermal and/or piezo jetting, such as by use of an ink jet device, for example. In one particular embodiment, a printing technique using a printer is used to fabricate a three-dimensional structure from thin, two-dimensional (“2D”) layers. A computer is used to generate cross-sectional patterns of the 2-D layers by storing a digital representation of the object in a computer memory. A computer-aided design or computer-aided manufacture (“CAM”) software is then used to section the digital representation of the structure into multiple, separate 2D layers. A printer, such as an inkjet printer, is used to fabricate a layer of structure precursor material for each layer sectioned by the software, onto a flat surface or support platform, optionally using a roller. The structure precursor material may be in the form of a liquid or a powder and may be, for example, a ceramic, metal, polymeric, or composite material. If the structure precursor material is in the form of a powder, a liquid binder is selectively deposited on the powder material using a printhead of the inkjet printer to produce areas of bound powder. The liquid binder, which is typically a polymeric resin or aqueous composition, is applied in the pattern of the cross-sectional pattern of the 2D layer. The liquid binder can penetrate gaps in the powder material and may react with the powder particles to create a layer bound in two dimensions. As the reaction proceeds, the binder also bonds each successive 2D layer to a previously deposited 2D layer. Additional 2D layers are formed by repeating the steps of depositing additional structure precursor material and applying the binder solution until the desired number of layers is produced. Since the liquid binder is selectively applied to the powder material, only certain areas of the powder material are bound within the layer and onto the previous layer. After the 3D object is formed, unbound powder is subsequently removed, e.g., by dissolving the powder in an appropriate solvent. The precursor structure may then be polymerized and/or crosslinked, and/or exposed to a precipitation medium to form the final porous structure.

In some embodiments, structures described herein are formed at least in part using a multi-photon lithography system. For instance, two-photon lithography or three-photon lithography systems may be used. Multi-photon polymerization may involve the use of an ultra-fast infrared laser (e.g., a femtosecond laser operating at a wavelength of 1028 nm), which can be focused into the volume of a structure precursor material including a photosensitive material. The polymerization process can be initiated by non-linear absorption within the focal volume. By moving the focused laser three-dimensionally through the resin, three-dimensional structures can be fabricated.

In one embodiment, a two-photon lithography system can be used at least in part to fabricate structures for tissue engineering and/or organ replacement. In a two-photon lithography system, a monomer mixed with a photo initiator that absorbs UV light may be exposed to an infra-red laser. Two photons of infra red light can be absorbed by the resin/chemicals and a single photon of ultra-violet light can be released. The released photon can then be absorbed by the photo initiator to produce free radicals which can cause polymerization of the monomers. Since the two-photon absorption cross-section is very small, for the release of sufficient UV light to induce free radical polymerization in the chemicals, a large amount of energy (terawatt) can be delivered to the chemical by the laser. This energy density could be generated at the focal point of a laser beam from an ultra-fast (e.g., femtosecond) pulse laser. Two-photon-absorption only occurs at the focal point of the beam and not at the laser beam path, hence a very small volume (e.g., femtoliter) of monomer can be polymerized through the release of free radicals from the photo initiator. After the structure has been polymerized, e.g., from a block of resin or in a petri dish of monomer, the unexposed chemicals can be washed away with a suitable solvent, leaving behind the final structure. The technique can been used with a variety of materials, including acrylate and epoxy polymers such as ethoxylated trimethylolpropane triacrylate ester and alkoxylated trifunctional acrylate ester, as described herein. This system can be used, for instance, when structures with fine resolution are desired. E.g., in some cases, multi-photon lithography can be used to form structures having submicron (e.g., less than one micron) resolution.

In one embodiment, stereolithography can be used at least in part to form structures for tissue engineering and/or organ replacement. Stereolithography may involve the use of a focused ultra-violet laser scanned over the top of a reservoir containing a photopolymerizable liquid polymer. The UV laser can cause the polymer to polymerize and/or crosslink where the laser beam strikes the surface of the reservoir, resulting in the formation of a solid or semi-solid polymer layer at the surface of the liquid. The solid layer can be lowered into the reservoir and the process can repeated for formation of the next layer, until a plurality of superimposed layers of the desired structure is obtained. This process may allow formation of various self-supporting structures, which may then be exposed to a precipitation medium to form cell growth template structures having a network uniform pores.

In another embodiment, selective laser sintering (or laser ablation) can be used at least in part to form structures for tissue engineering and/or organ replacement. Selective laser sintering may involve the use of a focused laser beam to sinter areas of a loosely-compacted plastic powder, where the powder is applied layer by layer. For instance, a thin layer of powder can be spread evenly onto a flat surface, e.g., using a roller mechanism. The powder can be raster-scanned using a high-power laser beam. The areas of the powder material where the laser beam was focused can be fused, while the other areas of powder can remain dissociated. Successive layers of powder can be deposited and raster-scanned, one on top of another, until an desired structure is obtained. In this process, each layer can be sintered deeply enough to bond it to the preceding layer.

In some embodiments involving three-dimensional fabrication, variation of the laser intensity and/or traversal speed can be used to vary the crosslinking density within a structure. In some cases, this allows the properties of the material to be varied from position to position with the structure. Variation of the laser intensity and/or traversal speed can also control the degree of local densification within the material. For instance, regions where the laser intensity is high or the traversal speed is low can create areas of higher density.

The following examples are intended to illustrate certain embodiments of the present invention, but are not to be construed as limiting and do not exemplify the full scope of the invention.

### Example 1

### Preparation of Polysulfone-Fullcure™ (PS-FC) Membranes

PS-FC membranes were prepared from polysulfone (PS, Sigma Aldrich, MW=26,000 g/mol) and Fullcure™ 700 monomer (Stratasys, USA) in weight ratios indicated in Table 1. The solvent, N,N-dimethylacetamide (DMAc, Sigma Aldrich), was used as received. FIG. 3 illustrates the synthesis scheme for the membrane preparation. A solution of PS in DMAc (10 wt %) and Fullcure™ 700 monomer was poured and sandwiched between two glass plates separated by a spacer that controlled the membrane thickness. Using this assembly, 80-micron-thick membranes were fabricated. The whole assembly was then subjected to UV curing for 30 min, which fixed the spatial arrangement of the polymer blend to form a free-standing structure. The structure was then immersed in a water bath to carry out a phase inversion process (a solvent-non-solvent treatment process, wherein water was used as the non-solvent (precipitation medium)) that led to the precipitation of PS to generate a porous network. After the membrane peeling away from the glass plate, the membrane was washed with distilled water and stored in distilled water at room temperature before use.

TABLE 1
Composition and properties of PS-FC membranes.

| Blend Composition | PS-FC-0.15 | PS-FC-0.2 | PS-FC-0.25 |
|---|---|---|---|
| PS (g) | 2.0 | 2.0 | 2.0 |
| FC (g) | 0.15 | 0.20 | 0.25 |
| Pore Diameter^{a} (μm) | 10-15 | 6-12 | 5-10 |
| Pore Diameter^{b} (nm) | 12.5 | 8.6 | 5.5 |
| MWCO (kDa) | 80 | 40 | 15 |
| Pure Water Flux | 717 | 597 | 161 |
| (L/m² · h) | | | |
| T_{g} (° C.) | 90, 195 | 95, 198 | 97, 200 |
| Contact Angle (°) | 30 | 35 | 38 |
| Storage Modulus | 1550 | 1920 | 2100 |
| Biocompatibility | Yes | Yes | Yes |

^{a}Measured by SEM.
^{b}Measured by MWCO.

### Example 2

### Characterization of PS-FC Membranes

The PS-FC membranes of Example 1 were characterized by scanning electron microscopy (JEOL JSM-7400F, 10 kV). The separation properties of the membranes were examined using the solute rejection technique for ultrafiltration membranes. The membranes were cut into the necessary size for use in the ultrafiltration cell.

The membranes were subjected to a pressure of 20 psi, and the flux of water through the membranes was measured under steady-state flow using Eq. 1:

$${J}_{w} = \frac{Q}{A Δ t} \qquad ( 1 )$$

where Q is the quantity of permeate collected (L), J_{w} is the water flux (L/m²·h), ΔT is the sampling time (h), and A is the membrane area (m²).

The pore size of the PS-FC membranes was determined by the ultrafiltration of polyethyleneglycol (PEG) with different molecular weights. A standard curve of the PEG solution was obtained using pure PEG fractions varying from 2 to 100 kDa. The molar masses of PEG were obtained by gel permeation chromatography. All the PEG solutions were prepared at a concentration of 1 wt %, and used as the feed. Higher concentrations were avoided since the permeate flux would decline with increasing feed concentration and affect the rejection performance. The MWCO values were calculated using Eq. 2:

$$\% S R = [ 1 - \frac{{C}_{p}}{{C}_{f}} ] \times 100 \qquad ( 2 )$$

where SR corresponds to 90% solute rejection and C_{f} and Cₚ are the feed and permeate concentrations (mol/dm³), respectively. The average pore radius r (Å) of the membrane was calculated from the MWCO value of the PEG by Eq. 3:

r=0.33(M)^{0.46} (3)

where M is molecular weight of solute.

The properties of the PS-FC membranes are summarized in Table 1. PS-FC membranes prepared with a PS:FC weight ratio smaller than 2:0.25 possessed finer and less interconnected pores. The PS-FC membranes showed two T_{g} values at ˜100 degrees Celsius and ˜200 degrees Celsius, and high storage modulus ranging from 1550 to 2100 MPa. A higher storage modulus was obtained for the membrane with a higher FC content, which provided a more elastic framework to the porous PS. The three membranes showed similar water Contact angles in the range of 30-38 degrees, indicating that the membranes could provide moderately wettable surfaces for the attachment and proliferation of tissue cells.

In all of the PEG rejection studies for determining the pore statistics and MWCO, the feed side was uniformly agitated to prevent concentration polarization and cake formation on the membrane surface, which would affect the flux, and ultimately, the partition coefficient and aggregate pore size.

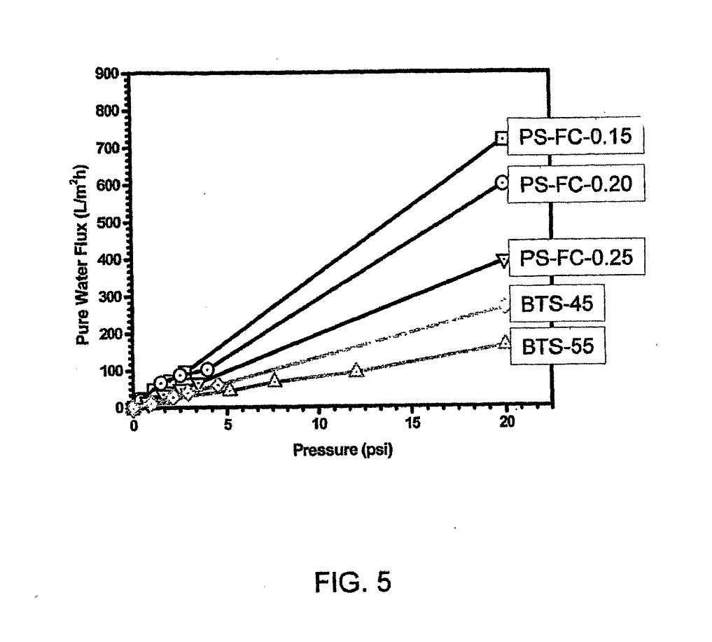

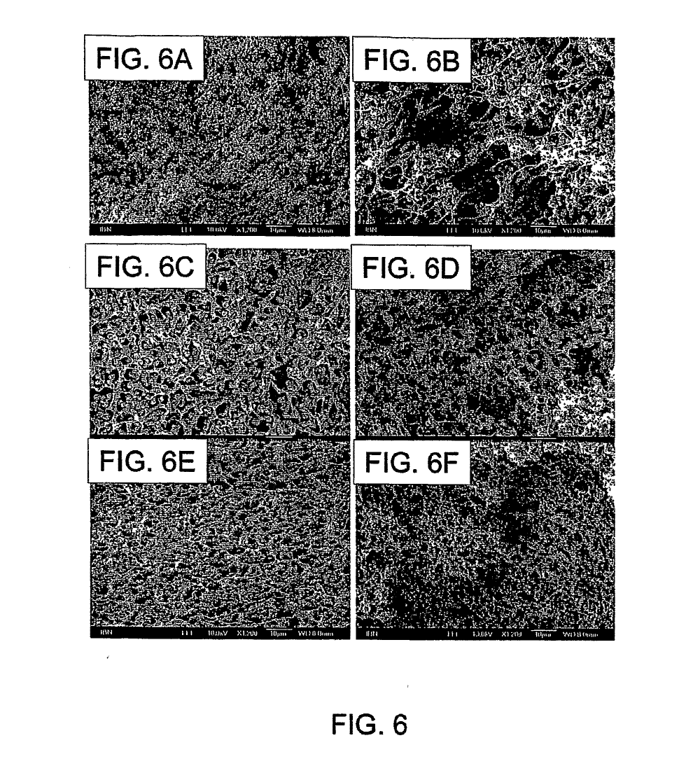

PS-FC-0.15, PS-FC-0.20 and PS-FC-0.25 blend membranes were subjected to water flux assessment at a pressure of 20 psi, and compared to the commercial BTS-45 and BTS-55 PS membranes (Pall Corporation, USA) having 0.3 and 0.2 micron sized pores, respectively. As shown in FIG. 5, a lower water flux was observed for PS-FC-0.25, compared to PS-FC-0.15 and PS-FC-0.20, which showed a steady-state flux of 717 and 597 L/m²h, respectively. The decrease hi flux with increasing FC could be attributed to the formation of smaller pores in the membranes. When the FC content was increased from 0.15 g to 0.25 g, the SEM pore diameter was significantly reduced from 10-15 μm to 5-10 μm. FIG. 6 shows SEM micrographs of top and cross-sectional views of the PS-FC membranes. In particular, FIGS. 6A (top) and 6B (cross-sectional) show images of PS-FC-0.15; FIGS. 6C (top) and 6D (cross-sectional) show images of PS-FC-0.20; and FIGS. 6E (top) and 6F (cross-sectional) show images of PS-FC-0.25. The pore diameters as calculated based on Eq. 3 in the MWCO study were much smaller than those observed under SEM (see Table 1). The measurements from MWCO experiments most likely gave a more accurate determination of pore size than SEM measurements. For the commercial BTS membranes, the SEM pore diameter was also found to be different than the pore diameter measured by MWCO.

In general, the PS-FC membranes showed a transition in permeation properties from microfiltration to ultrafiltration range with increasing FC content. Their cut-off curves were sharper compared to that of the commercial BTS-45 and BTS-55 PS membranes. These findings indicated that the PS-FC membranes are promising materials as porous separation membranes.

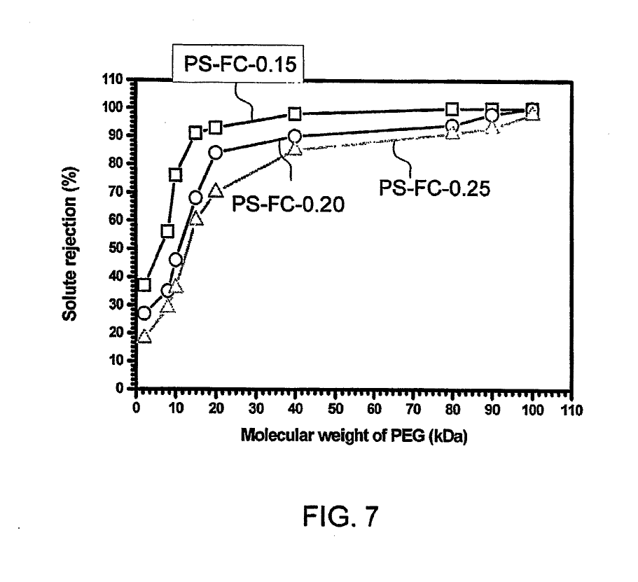

The typical PEG rejection curves for the PS-FC membranes as a function of PEG molecular weight are shown in FIG. 7. The cut-off level was measured based on 90% rejection of PEG of a particular molecular weight. In general, the cut-off level of the membrane corresponded to its mean pore size.

The mean pore sizes were determined from the cut-off values as measured at 90% rejection for PEG molecules. The increase in MWCO with decreasing FC content indicated an increase in pore size.

This example shows that membranes having uniform pore sizes can be fabricated according to methods described herein.

### Example 3

### Growth of Cells on PS-FC Membranes

This example shows that the PS-FC membranes of Example 1 are compatible with living cells. For the biocompatibility study, MDCK (Madin-Darby kidney cells) were plated on the PS-FC membranes (without coating the membranes with cell adhesion proteins), and were cultured in a humidified incubator with 5 vol % CO₂. The cell viability and proliferation were examined with optical fluorescent microscopy using DAPI staining.

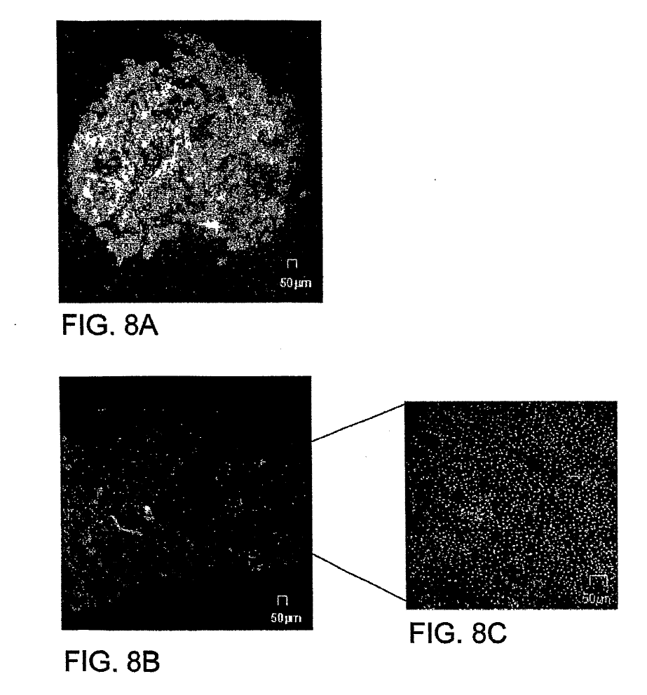

The morphology of the MDCK cells was studied after 4 days of culture on a PS-FC-0.25 membrane. FIG. 8 shows DAPI staining of the nuclei of the living cells. FIGS. 8B and 8C shows that the MDCK cells adhered to the membrane, and covered the membrane surface homogeneously as a monolayer, as is characteristic of renal tubule cells, even without the use of cell adhesion proteins. In contrast, FIG. 8A shows the adhesion of cells in the form of clusters on a commercial polysulfone SUPOR_{—}1200 membrane (Pall Corporation, USA) when the membrane did not include a layer of cell adhesion proteins. Thus, it was concluded that the PS-FC membrane provided a non-toxic substrate for a better culture of a monolayer of MDCK cells, compared to the SUPOR_{—}1200 membrane.

This example shows that PS-FC membranes can serve as bioactive membranes without further processing, such as coating the membranes with cell adhesion proteins. This represents an improvement compared to existing polymer surfaces used for bioartificial purposes, since many conventional polymer membranes fail to show cell adherence without ECM surface coatings. This example also suggests that PS-FC materials may be suitable for application in biohybrid artificial organ devices.

### Example 4

Fabrication of 3D Porous Structures

In this prophetic example, a 3D porous structure suitable for use as a template for cell growth is fabricated. A CT scan of a tissue and/or organ of a patient is converted into a CAD file and fed into a three-dimensional printer. A structure precursor material is prepared by mixing Fullcure™ 700 monomer, polysulfone, and N,N-dimethylacetamide solvent, to form a homogeneous solution. The structure precursor material is introduced into the three-dimensional printer, as is a sacrificial material (e.g., Fullcure™ 705 support) for forming open areas (e.g., cavities) in the structure. The structure precursor material and sacrificial material are dispensed droplet by droplet and layer by layer onto a substrate. After several layers of the precursor structure are deposited, the precursor structure is subjected to UV radiation for a sufficient period of time to cause polymerization (and/or crosslinking) of the Fullcure™ monomer. The resulting precursor structure is immersed in a water bath, which causes precipitation of the polysulfone and removal of the N,N-dimethylacetamide solvent from the structure. As a result of this process, a uniform network of pores is formed in the structure. The structure is then immersed in 25% tetra methyl ammonium hydroxide (TMAH) solution until the sacrificial material has been removed from the structure. The structure is then used as a template for cell growth where living cells can be immobilized and perform their normal physiological functions.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of”, when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of fabricating a structure for use as a template for cell growth, comprising:
dissolving at least first and second polymer components in a precursor solvent to form a structure precursor material;
shaping the structure precursor material into a structure suitable for use as a template for cell growth;
crosslinking the first polymer component; and
removing at least a portion of the precursor solvent from the structure, thereby forming a plurality of pores in the structure.

2. A method as in claim 1, further comprising contacting the structure with a precipitation medium and removing the portion of the precursor solvent in the precipitation medium.

3. A method as in claim 2, wherein the precipitation medium is a solvent.

4. A method as in claim 2, wherein the precipitation medium is water.

5. A method as in claim 2, wherein the precipitation medium is air.

6. A method as in claim 1, wherein the precursor solvent is non-reactive with the first and second polymer components.

7. A method as in claim 2, wherein the second polymer component is substantially non-crosslinked after the crosslinking step.

8. A method as in claim 1, wherein the plurality of pores have an average pore size of less than or equal to 20 microns formed in at least a portion of a wall of the structure.

9. (canceled)

10. A method as in claim 1, wherein no more than about 5% of all pores deviate in size from the average pore size of the plurality of pores by more than about 20%.

11. (canceled)

12. A method as in claim 1, wherein greater than 90% of the pores have a molecular weight cutoff of about 80 kDa.

13-14. (canceled)

15. A method as in claim 1, wherein shaping the structure precursor material comprises three-dimensional printing.

16. A method as in claim 1, further comprising exposing the structure to an environment facilitating cell growth onto the structure.

17. A method as in claim 1, further comprising exposing the structure to an environment facilitating cell ingrowth into pores of the structure.

18-19. (canceled)

20. A method as in claim 1, wherein the first polymer component is an acrylic-based monomer.

21. A method as in claim 1, wherein the second polymer component is a sulfone-based monomer.

22. A method of fabricating a structure for use as a template for cell growth, comprising:
providing a structure precursor material comprising at least first, second, and third components;
shaping the structure precursor material into a structure suitable for use as a template for cell growth;
crosslinking the first component;
precipitating the second component in a precipitation medium; and
removing the third component from the structure in the precipitation medium, thereby forming a plurality of pores in the structure.

23-33. (canceled)

34. A method of fabricating a structure for use as a template for cell growth, comprising:
mixing at least first and second polymer components in a precursor solvent to form a homogeneous structure precursor material, wherein the first and second polymer components and the precursor solvent are miscible at 25 degrees Celsius and 1 atm;
printing the structure precursor material to form a three-dimensional structure suitable for use as a template for cell growth; and
removing the precursor solvent from the structure, thereby forming a plurality of pores in the structure.

35-41. (canceled)

42. A method of fabricating a structure for use as a template for cell growth, comprising:
forming a cell growth template precursor structure comprising at least first and second polymer components and a fluid carrier;
crosslinking the first polymer component thereby forming a self-supporting structure;
removing at least a portion of the fluid carrier from the self-supporting structure, thereby forming a plurality of pores in the structure suitable for templated cell growth,
wherein the porous structure is formed in a shape suitable for templated cell growth.

43-46. (canceled)

47. An article for use as a template for cell growth, comprising:
a structure comprising at least one wall defining a cavity; and
a plurality of pores having an average pore size of less than or equal to 20 microns formed in at least a portion of the wall, wherein no more than about 5% of all pores deviate in size from the average pore size of the plurality of pores by more than about 20%,
wherein the structure is constructed and arranged for use as a template for cell growth.

48-56. (canceled)

57. A method of fabricating a structure for use as a template for cell growth, comprising:
dissolving at least first and second polymer components in a precursor solvent to form a structure precursor material;
shaping the structure precursor material into a structure suitable for use as a template for cell growth;
exposing the structure precursor material to UV radiation; and
removing at least a portion of the precursor solvent from the structure, thereby forming a plurality of pores in the structure.

58-60. (canceled)
